(19) United States
(12) Patent Application Publication
(10) Pub. No.: US 2003/0195607 A1
(43) Pub. Date: Oct. 16, 2003

(54) Method and apparatus to attach an unsupported surgical component

(76) Inventors: Hugh H. Trout III, Washington, DC (US); Frank Patterson, Exeter, NH (US)

Correspondence Address: COLLIER SHANNON SCOTT, PLLC, 3050 K STREET, NW, SUITE 400, WASHINGTON, DC, 20007, US

(21) Appl. No.: 10/389,972

(22) Filed: Mar. 18, 2003

Related U.S. Application Data
(60) Provisional application No. 60/364,601, filed on Mar. 18, 2002.
(60) Provisional application No. 60/382,084, filed on May 22, 2002.
(60) Provisional application No. 60/374,833, filed on Apr. 24, 2002.
(60) Provisional application No. 60/375,807, filed on Apr. 29, 2002.

(51) Int. Cl. A61F 2/06

(52) U.S. Cl. 623/1.13

(57) ABSTRACT

An insertion apparatus comprises an introducer sheath, a surgical component, at least two attachment wires, and a longitudinal wire. The insertion apparatus is used for deploying a surgical component to a site in a vessel. The introducer sheath has a lumen, a proximal opening and a distal opening with an appropriate diameter and length allowing fro insertion and navigation through the vessel. The surgical component has a tube portion with a top and at least one limb. The surgical component corresponds to the site in the vessel and fits within the lumen of the introducer sheath. The attachment wires have a first and a second end. The first end is releasably connected to the top of the surgical component and the second end converges above the top of the surgical component. The activation of a releasing mechanism releases the first end from the top of the surgical component. The longitudinal wire anchors from the second end extending through the surgical component and through the introducer sheath to the releasing mechanism outside the vessel.

### CROSS-REFERENCE TO RELATED APPLICATIONS

The present invention relates to, and is entitled to the benefit of the earlier filing date and priority of U.S. Application No. 60/364, 601 filed Mar. 18, 2002, U.S. Application No. 60/382,084, filed May 22, 2002; U.S. Application No. 60/374,833 filed Apr. 24, 2002; and U.S. Application No. 60/375,807 filed Apr. 29, 2002.

### FIELD OF THE INVENTION

The present invention relates to apparatus and methods for performing a surgical procedure. More particularly, the present invention relates to apparatus and methods to attach an unsupported surgical component.

### BACKGROUND OF THE INVENTION

An aneurysm is a ballooning of the wall of an artery resulting from the weakening of the artery due to disease or other conditions. Left untreated, the aneurysm will frequently rupture, resulting in loss of blood through the rupture and death.

Aortic aneurysms are the most common form of arterial aneurysm and are life threatening. The aorta is the main artery which supplies blood to the circulatory system. The aorta arises from the left ventricle of the heart, passes upward and bends over behind the heart, and passes down through the thorax and abdomen. Among other arterial vessels branching off the aorta along its path, the abdominal aorta supplies two side vessels to the kidneys, the renal arteries. Below the level of the renal arteries, the abdominal aorta continues to about the level of the fourth lumbar vertebrae (or the navel), where it divides into the iliac arteries. The iliac arteries, in turn, supply blood to the lower extremities and perineal region.

It is common for an aortic aneurysm to occur in that portion of the abdominal aorta between the renal arteries and the iliac arteries. This portion of the abdominal aorta is particularly susceptible to weakening, resulting in an aortic aneurysm. Such an aneurysm is often located near the iliac arteries. An aortic aneurysm larger than about 5 cm in diameter in this section of the aorta is ominous. Left untreated, the aneurysm may rupture, resulting in rapid, and usually fatal, hemorrhaging. Typically, a surgical procedure is not performed on aneurysms smaller than 5 cm because no statistical benefit exists in performing such procedures.

Aneurysms in the abdominal aorta are associated with a particularly high mortality rate; accordingly, current medical standards call for urgent operative repair. Abdominal surgery, however, results in substantial stress to the body. Although the mortality rate for an aortic aneurysm is high, there is also considerable mortality and morbidity associated with open surgical intervention to repair an aortic aneurysm. This intervention involves penetrating the abdominal wall to the location of the aneurysm to reinforce or replace the diseased section of the aortic aneurysm. A prosthetic device, typically a synthetic tube graft, is used for this purpose. The graft serves to exclude the aneurysm from the circulatory system, thus relieving pressure and stress on the weakened section of the aorta at the aneurysm.

Repair of an aortic aneurysm by surgical means is a major operative procedure. Substantial morbidity accompanies the procedure, resulting in a protracted recovery period. Further, the procedure entails a substantial risk of mortality. While surgical intervention may be indicated and the surgery carries attendant risk, certain patients may not be able to tolerate the stress of intra-abdominal surgery. It is, therefore, desirable to reduce the mortality and morbidity associated with intra-abdominal surgical intervention.

In recent years, methods have been developed to attempt to treat an aortic aneurysm without the attendant risks of intra-abdominal surgical intervention. Among them are inventions disclosed and claimed in Kornberg, U.S. Pat. No. 4,562,596 for Aortic Graft, Device and Method for Performing an Intraluminal Abdominal Aortic Aneurysm Repair; Lazarus, U.S. Pat. No. 4,787,899 for Intraluminal Graft Device, System and Method; and Taheri, U.S. Pat. No. 5,042,707 for Intravascular Stapler, and Method of Operating Same.

Although in recent years certain techniques have been developed that may reduce the stress, morbidity, and risk of mortality associated with surgical intervention to repair aortic aneurysms, none of the systems that have been developed effectively treat the aneurysm and exclude the affected section of aorta from the pressures and stresses associated with circulation. None of the devices disclosed in the references provide a reliable and quick means to reinforce an aneurysmal artery. In addition, all of the prior references require a sufficiently large section of healthy aorta surrounding the aneurysm to ensure attachment of the graft. The neck of the aorta at the cephalad end (i.e., above the aneurysm) is usually sufficient to maintain a graft's attachment means. However, when an aneurysm is located near the iliac arteries, there may be an ill-defined neck or no neck below the aneurysm. Such an ill-defined neck would have an insufficient amount of healthy aortic tissue to which to successfully mount a graft. Furthermore, much of the abdominal aorta wall may be calcified which may make it extremely difficult to attach the graft to the wall. Furthermore, the prior art does not disclose surgical devices that can be used during a surgical procedure that address these concerns. Others have developed devices that are not easily manipulated or oriented during intraluminal surgical procedures.

It is therefore an advantage of some, but not necessarily all, embodiments of the present invention to provide an improved apparatus and method to attach an unsupported surgical component.

Additional advantages of various embodiments of the invention are set forth, in part, in the description that follows and, in part, will be apparent to one of ordinary skill in the art from the description and/or from the practice of the invention.

### SUMMARY OF THE INVENTION

Responsive to the foregoing challenges, Applicant has developed an innovative method and apparatus to attach an unsupported surgical component.

An embodiment of the present invention is an insertion apparatus for deploying a surgical component to a site in a vessel comprising: attachment means for attaching the surgical component to the insertion apparatus, wherein the attachment means is releasably connected to the surgical component; and release means for releasing the attachment means from the surgical component, wherein the release means is in communication with the attachment means.

An embodiment of the present invention is an insertion apparatus for deploying a surgical component to a site in a vessel comprising: at least one attachment wire with a first end and a second end; a support wire; wherein the first end of the at least one attachment wire is releaseably connected to the surgical component and the second end is connected to the support wire; and wherein the attachment wire and support wire positions the surgical component at a site in the vessel.

An embodiment of the present invention is an insertion apparatus for deploying a surgical component to a site in a vessel comprising: an attachment hoop releasably connected to the surgical component; at least one attachment wire with a first end and a second end; a support wire; wherein the first end of the at least one attachment wire is connected to the attachment hoop and the second end of the at least one attachment wire is connected to the support wire; and wherein the insertion apparatus positions the surgical component in the vessel.

An embodiment of the present invention is an insertion system for deploying a surgical component to a site in a vessel comprising: a surgical component; an insertion apparatus; attachment means for attaching the surgical component to the insertion apparatus, wherein the attachment means is releasably connected to the surgical component; and release means for releasing the attachment means from the surgical component, wherein the release means is in communication with the attachment means.

An embodiment of the present invention is a method for positioning a surgical component to a site in a vessel comprising the steps of: introducing an insertion apparatus proximal to the site in the vessel; activating the insertion apparatus; and withdrawing the insertion apparatus. An embodiment of the present invention further comprises the step of fastening the surgical component to the vessel.

In accordance with an embodiment of the present invention, there is provided an introducer sheath for deploying a surgical component to a site in a vessel. The apparatus includes an introducer sheath, a surgical component, at least two attachment wires, and a longitudinal wire. The introducer sheath has a lumen, a proximal opening and a distal opening with an appropriate diameter and length allowing for insertion and navigation through the vessel. The surgical component has a tube portion with a top and at least one limb. The surgical component corresponds to the site in the vessel and fits within the lumen of the introducer sheath. The attachment wires have a first and a second end. The first end is releasably connected to the top of the surgical component and the second end converges above the top of the surgical component. The activation of a releasing mechanism releases the first end from the top of the surgical component. The longitudinal wire anchors from the second end extending through the surgical component and through the introducer sheath to the releasing mechanism outside the vessel.

In accordance with an embodiment of the present invention, there is provided a method for attaching a surgical component with an insertion apparatus to site in a vessel comprising the steps of: inserting the insertion apparatus to the site in the vessel; positioning the surgical component to the site in the vessel; fastening the surgical component to the vessel; activating a release mechanism of the insertion apparatus; and withdrawing the insertion apparatus.

It is to be understood that both the foregoing general description and the following detailed description are exemplary and explanatory only, and are not restrictive of the invention as claimed. The accompanying drawings, which are incorporated herein by reference, and which constitute a part of this specification, illustrate certain embodiments of the invention and, together with the detailed description, serve to explain the principles of the present invention.

### BRIEF DESCRIPTION OF THE DRAWINGS

In order to assist the understanding of this invention, reference will now be made to the appended drawings, in which like reference characters refer to like elements. The drawings are exemplary only, and should not be construed as limiting the invention.

FIGS. 1 and 2 are perspective views of a surgical component with and without distinctive markings according to an embodiment of the present invention.

FIG. 3 is a perspective view of a surgical component according to an embodiment of the present invention.

FIGS. 4, 5, and 6 are enlarged perspective views of embodiments of a release mechanism according to embodiments of the present invention.

FIG. 7 is a perspective view of an embodiment of the attachment wires secured to a bulb with a subsequent device attached thereto.

FIG. 8 is a perspective view of an expanded surgical component with an insulated longitudinal wire according to an embodiment of the present invention.

FIG. 9 is a perspective view of a contracted surgical component according to an embodiment of the present invention.

FIG. 10 is a perspective view of an embodiment of the surgical component attached to a release mechanism employing an electrical current to separate the attachment wires.

FIG. 11 is a perspective view of an embodiment of the surgical component having distinctive markings and sutures attached to the ends of the limbs.

FIGS. 12 and 13 are perspective views of the graft packed with a short sheath, which may be flared according to an embodiment of the present invention.

FIGS. 14, 15, and 16 are perspective views of the graft with limbs trimmed to an appropriate length by the inserter to accommodate patient size according to an embodiment of the present invention.

FIGS. 17 and 18 are perspective views of the introducer sheath with a detachable hemostatic valve and a dilator according to an embodiment of the present invention.

FIG. 19 is a perspective view of introducer sheath and the short sheath wherein the short sheath containing the graft is introduced into the introducer sheath according to an embodiment of the present invention.

FIG. 20 is a perspective view of the graft advanced to the area just before the soft area of the introducer sheath where the graft is entirely removed from the short sheath according to an embodiment of the present invention.

FIGS. 21 and 22 are perspective views of the graft with the short sheath removed and the expansion of the soft or resilient area according to an embodiment of the present invention.

FIG. 23 is a perspective view of the graft advanced to the end of the introducer sheath according to an embodiment of the present invention.

FIGS. 24 and 25 are perspective views of a long delivery catheter and a short sheath with a graft according to an embodiment of the present invention.

FIGS. 26 and 27 are perspective views of the top of the short sheath being placed at the opening of the long delivery catheter according to an embodiment of the present invention.

FIG. 28 is a perspective view of the entire graft assembly advanced into the long delivery catheter according to an embodiment of the present invention.

FIG. 29 is a perspective view of an embodiment of the insertion apparatus within an aneurysm.

FIGS. 30 and 31 are perspective views of an embodiment of the insertion apparatus with a snare catheter within an aneurysm.

FIGS. 32 and 33 are perspective views of the introducer sheath pulled back from the graft in the aneurysm according to an embodiment of the present invention.

FIG. 34 is a perspective view of the unsupported graft within the aortic neck with the attachment wires unrestrained according to an embodiment of the present invention.

FIG. 35 is a perspective view of the snare catheter passed through the left femoral artery out to the right femoral artery according to an embodiment of the present invention.

FIGS. 36 and 37 are perspective views of the aneurysm with graft limbs in the right femoral artery and the left femoral artery according to an embodiment of the present invention.

FIG. 38 is a perspective view of a fastener delivery catheter inserted and positioned within the graft located in the aortic neck according to an embodiment of the present invention.

FIG. 39 is a perspective view of the graft with fasteners inserted around the top portion of the graft according to an embodiment of the present invention.

FIGS. 40 and 41 are perspective views releasing the attachment wires from a graft with the fasteners inserted according to an embodiment of the present invention.

FIG. 42 is a perspective view of the snare catheter inserted above the attachment wires according to an embodiment of the present invention.

FIGS. 43 and 44 are perspective views of the snare catheter tightened around the attachment wires for removal according to an embodiment of the present invention.

FIG. 45 is a perspective view of the graft in the aneurysm without the attachment wires according to an embodiment of the present invention.

FIG. 46 is a perspective view of two stents inserted in the right and left limbs of the graft according to an embodiment of the present invention.

FIG. 47 is a perspective view of the sutures attached to the right and left limbs of the graft which have been cut by a suture cutter according to an embodiment of the present invention.

FIG. 48 is a perspective view of an embodiment of the longitudinal wire with a containment sheath according to an embodiment of the present invention.

FIG. 49 is a perspective view of the containment sheath enclosing the attachment wires of the graft according to an embodiment of the present invention.

FIG. 50 is a perspective view of an alternate embodiment of the short sheath with an open or partial sheath attached according to an embodiment of the present invention.

FIG. 51 is a perspective view of an insertion sheath with a short sheath according to an embodiment of the present invention.

FIG. 52 is a perspective view of an alternate embodiment of the short sheath with a closed circumferential sheath according to an embodiment of the present invention.

FIGS. 53 and 54 are perspective views of the graft when a release mechanism is applied according to an embodiment of the present invention.

FIGS. 55 and 56 are perspective views of the constraining and releasing of the attachment wires according to an embodiment of the present invention.

FIG. 57 is a perspective view of a method to attach and detach the tube portion of the graft from the attachment wires according to an embodiment of the present invention.

FIG. 58 is a perspective view of the tube portion of a graft contained within the circumferential short sheath according to an embodiment of the present invention.

FIG. 59 is a perspective view of the introducer sheath having a plurality of soft resilient areas and a hemostatic valve according to an embodiment of the present invention.

FIG. 60 is a perspective view of introducer sheaths having a variety of connectors and hemostatic valves according to an embodiment of the present invention.

FIG. 61 is a perspective view of multiple ports within the inner wall of the introducer sheath according to an embodiment of the present invention.

FIG. 62 is a cross section view of the introducer sheath with a passageway according to an embodiment of the present invention.

FIG. 63 is a perspective view of an introducer sheath containing a plurality of soft spots attached by a connector to a double port also having a plurality of soft spots according to an embodiment of the present invention.

FIG. 64 is a perspective view of the introducer sheath having a user-controlled variable restricting device according to an embodiment of the present invention.

FIGS. 65 and 66 are perspective views of a suture cutter with a suture according to an embodiment of the present invention.

FIG. 67 is a perspective view of an embodiment of a closed circumferential sheath with a partial or open sheath according to an embodiment of the present invention.

### DETAILED DESCRIPTION OF EMBODIMENTS OF THE INVENTION

Reference will now be made in detail to embodiments of the present invention, examples of which are illustrated in the accompanying drawings.

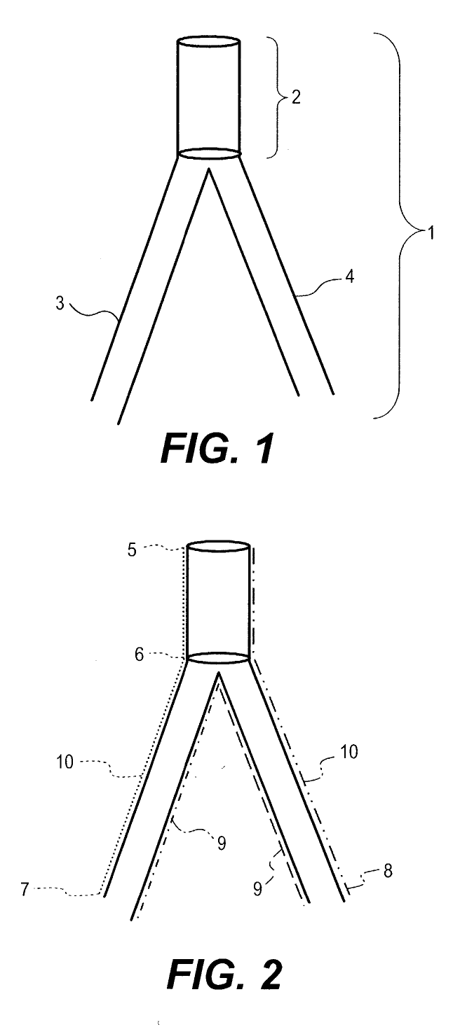

The following methods and apparatus may be used with any suitable surgical components. By way of example only, the following paragraphs describe methods of use and the apparatus with surgical components, such as, but not limited to, grafts, prosthetic grafts, endografts, patches, or any other suitable surgical component. For purposes of explanation only, these surgical components may be on-the-shelf ready for use or custom fabricated. The surgical components may have the tube portion 2, as shown in FIG. 1, with fixed length. The surgical component may be a patch or tubular in shape, and may or may not have limbs. If a custom made surgical component were required, it is likely to be in an unusual circumstance and that would be known in advance (because of the preoperative three dimensional (3D) computerized tomography (CT) scan or any other appropriate scan or modality) and a special order could be placed. The following examples are explanatory only and not meant to be limiting of the type of surgical component that may be used.

FIG. 1 depicts a surgical component 1, which may be, for purposes of example only, and is not limited to, a prosthetic bifurcated graft 1. This surgical component may have a fixed length tube portion 2 of any suitable size and may be, but is not limited to, from about 1-10 cm and may be comprised of, but is not limited to, Dacron, polyester, or PTFE. Graft limbs 3 and 4 may be supplied along with the intent that they will be trimmed to fit the patient size.

Depending on the size of the vessel in the patient, each tube diameter may be supplied with three different limb diameters. For example, the middle diameter may be one half of the tube diameter and the two others may be about 2 mm less and about 2 mm more than half the diameter of the tube portion. Thus, a 20 mm tube may come in three versions: 1) 20 mm tube and 8 mm limb diameter; 2) 20 mm tube and 10 mm limb diameter; and 3) 20 mm tube and 12 mm limb diameter. It is also possible to have one limb diameter larger than the other.

In FIG. 2, distinctive markings depicted by rings, dots and dashes may be added to the graft. Distinctive markings may be comprised of radiopaque markers or any other suitable distinctive markings, radiopaque or not, to facilitate easy identification under imaging techniques, including, but not limited to, fluoroscopy, magnetic resonance (MR), or any other suitable imaging modality. Radiopaque markers with surgical components are described in U.S. application Ser. No. 10/173,028 filed Jun. 18, 2001, the entire disclosure and subject matter of which is hereby incorporated herein by reference. These markers may be small pieces of radiopaque material or may be vaporized radiopaque material imbedded into the graft material, or of any other suitable material and affixed by any other suitable method. These markers will allow the inserter to locate the top 5, the bifurcation 6, and the ends of the limbs 7 and 8. These markers will also enable the inserter to distinguish among the medial 9 and lateral 10 portions of the graft limbs.

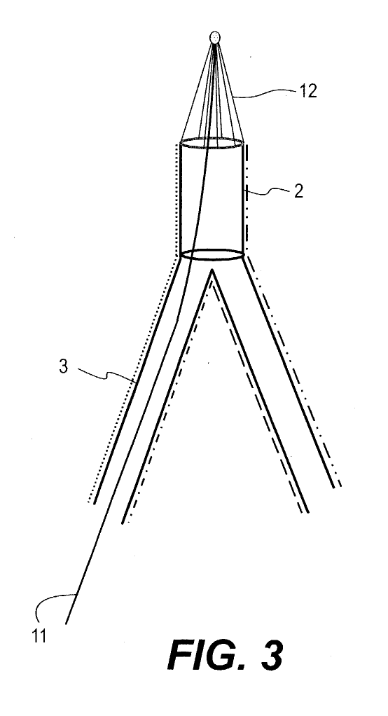

In FIG. 3, attachment means may comprise a support wire 11, attachment wire 12, and may further comprise additional support structures, including, but not limited to, at least one ring or hoop structure. Support wire 11 may have a slight bend or a tip deflection capability and may be inserted through the left 4 or right 3 limb of the graft 1 up through the tube portion 2 of the graft 1. The support wire 11 may be insulated. In FIGS. 3-6, a plurality of attachment wires 12 are connected to the graft 1 and to the support wire 11, either directly or through an intermediate structure, such as, but not limited to, a ring or hoop, described in more detail below. The attachment wires 12 may be connected to the top 5 of the graft 1 shown,in FIG. 2.

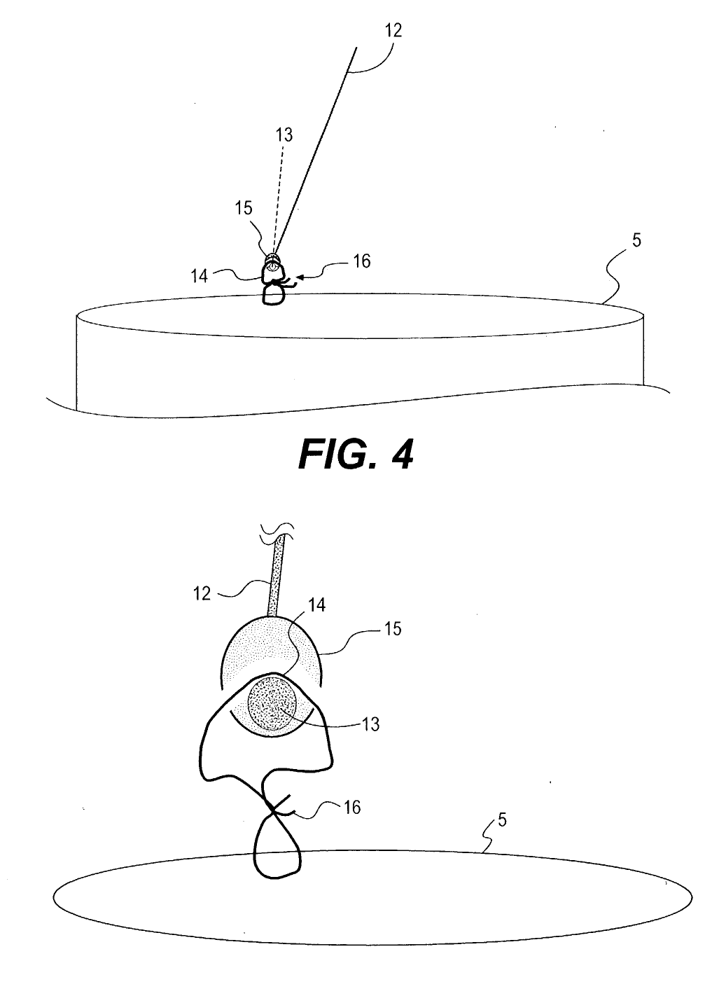

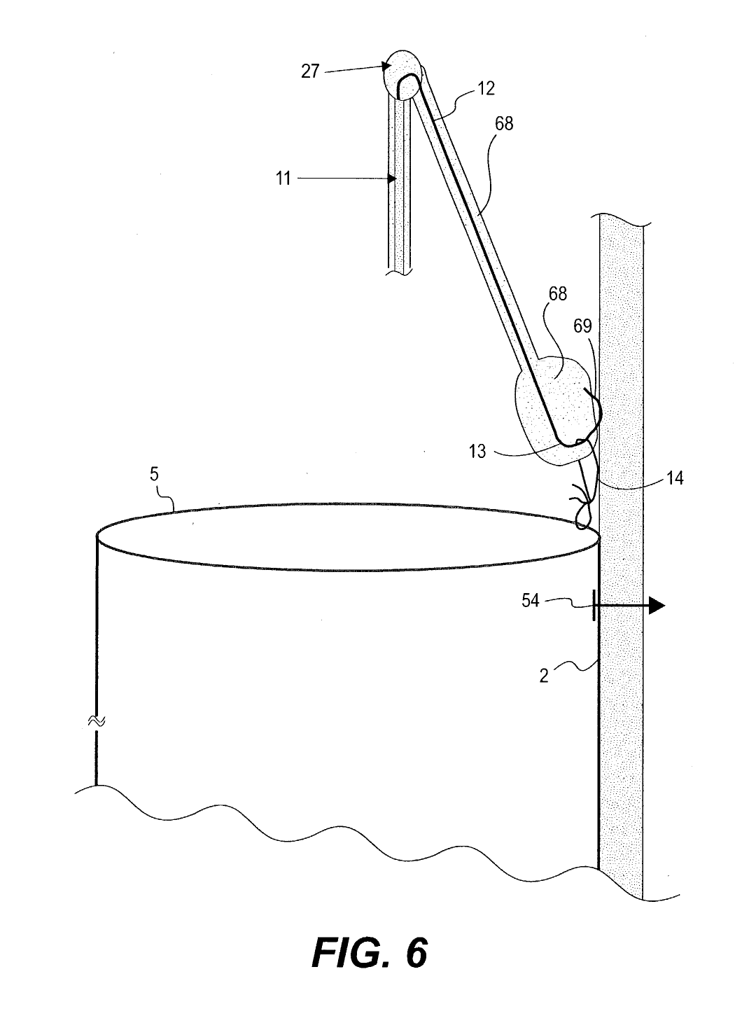

Details of the attachment wires 12 are shown in three blow-up drawings, FIGS. 4, 5, and 6. In one embodiment, an electric current is applied to the attachment wires 12. Attachment wires 12 connect to a filament 13 and/or a ball 15 that increases in temperature when a current is supplied. The filament 13 heats and burns through the suture 14 releasing the attachment wires from the graft 3. Release means may comprise an electrical current, heat, vibration, laser, dissolvable sutures, adhesive, metal alloys, or any other suitable release mechanism. In the embodiment shown in FIGS. 4 and 5, the suture 14 is cut by the hot filament 13 and separates from the ball 15. Because of the method used to tie the sutures, suture fragments are discouraged from embolizing elsewhere. FIG. 6 depicts another embodiment of the release mechanism where the attachment wires 12 are insulated with any suitable material 68. When a current is applied to the insulated 68 attachment wires 12, the filament 13 burns and severs the suture 14 attached to the top 5 of the graft 1. An exposed portion of the filament 13 may be used to ground the wire through tissue, such as the aortic wall. Multiple different circuit designs may be selected. For instance, instead of using a patient ground (electrical return path), the ground can be provided with a separate conductor onboard the device. Additionally, a feedback circuit, or any other suitable means, may be added to indicate to the user when the separation had been successfully completed.

A knot 16 of the suture 14 may be placed above the graft to decrease the bulkiness of the graft especially as the graft is being pushed/pulled through the introducer sheath 95. The balls 15 located near the top 5 of the graft 1 can be at different levels in order to reduce the bulk in any one transverse plane. Moreover, this method of attachment/release can be used in holding and detaching any foreign material in any vessel, artery or vein or in any tissue where a suture is a temporary attachment mechanism. A similar release mechanism could be achieved with magnets, glues that dissolve upon electrical or heat stimulation, or metal alloys that separate in response to heat or electrical current, or any other suitable method.

It will be apparent to those skilled in the art that variations and modifications of the present invention can be made without departing from the scope or spirit of the invention. For instance, as the need to insert objects into body orifices (whether percutaneously, through small incisions, or endoscopically) increases, it is progressively more important to reduce the size of these objects during their insertion as much as possible. One way to accomplish this objective is to insert components of the object through a small opening into the area where they will be attached or deployed. Once the components are inserted they may then be attached to each other to form a larger object. This may be accomplished via a scope or sheath inserted into an open orifice, such as, but not limited to, the trachea, the esophagus, the anus, the nose, the vagina or the urethra. Likewise, it may be done through an incision or skin penetration into the chest, abdomen, soft tissue, or a joint. It also may be accomplished through an incision or percutaneous penetration into an artery or vein remote from the desired site of implantation.

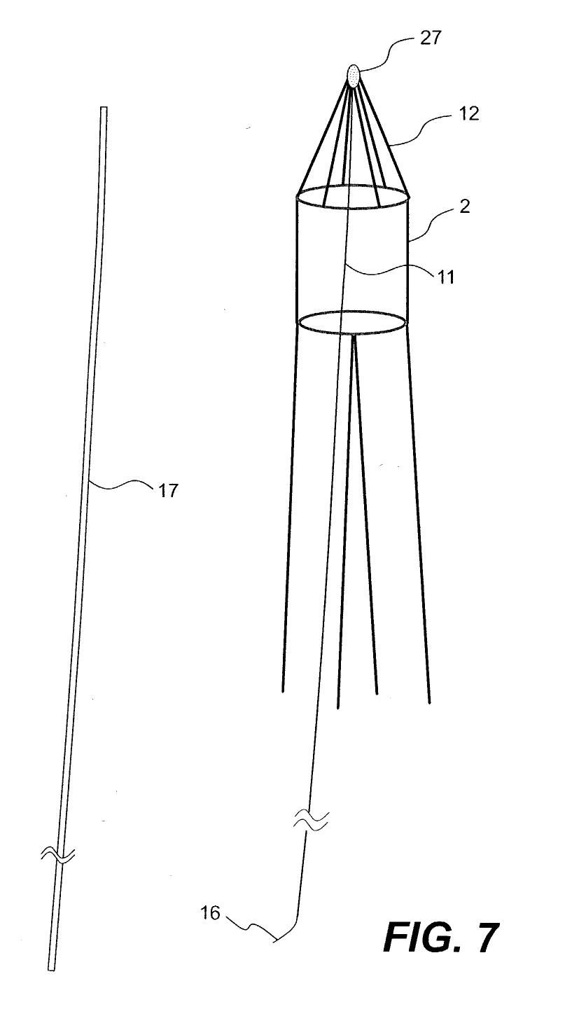

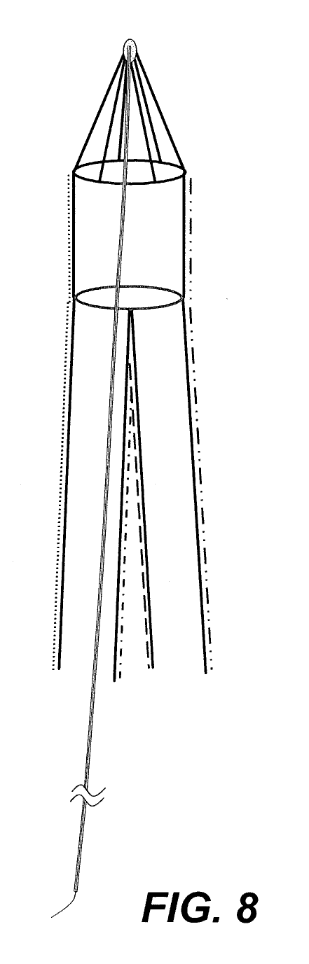

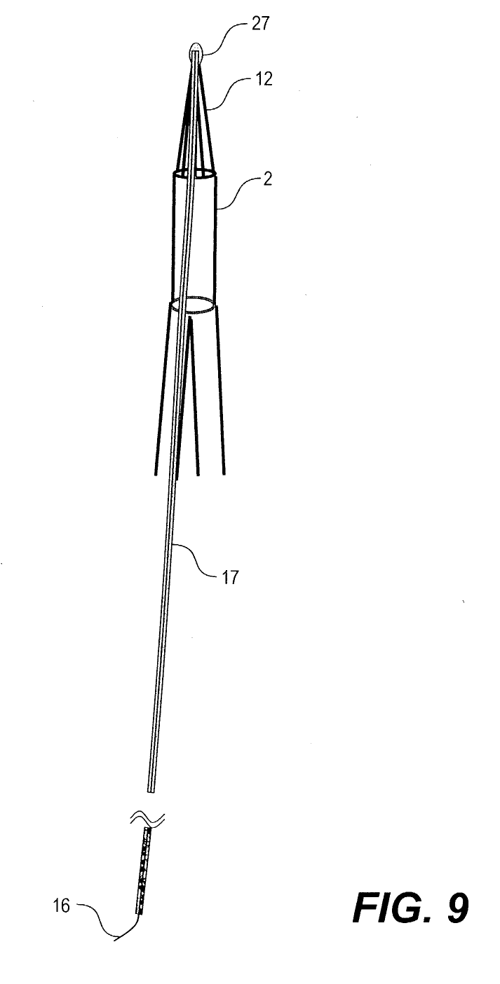

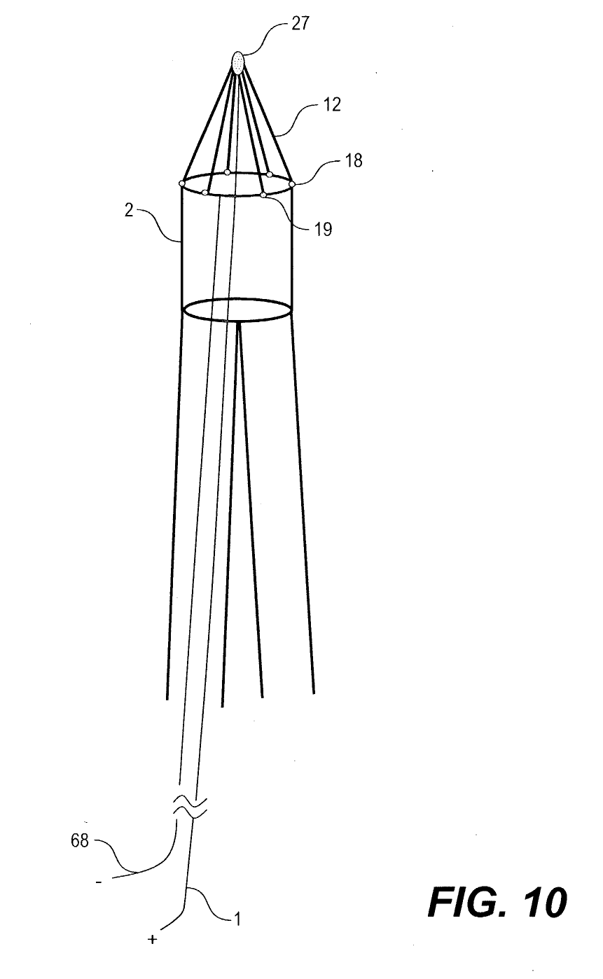

As an example of such an approach, an embodiment shown in FIG. 7, a very fine support wire 11, which may be, but is not limited to, a wire, a flexible suture, or any other suitable object is attached to a bulb 27 to which attachment wires 12 are connected on one and, on the other, to a graft 1 or the tube portion 2, such as, but not limited to, a prosthetic graft. This entire device may be compressed and delivered into an aorta through a small catheter. The wire 11 may have a smaller diameter so as to reduce the size of the device as it is being inserted. It may be necessary, however, for the device to be manipulated in such a way that the smaller diameter wire 11 is not capable of facilitating. In such an instance, it may be desirable to introduce over, about, or adjacent to the wire 11 at the distal end 16 a subsequent device 17 that attaches to a part of the bulb 27 so as to provide capabilities not provided by the small diameter wire 11. For instance, the subsequent device 17 may provide more support for the device, insulation for the wire 68 as in FIG. 8, or some sort of manipulating system that allows torsion or positioning control or control of the angulation of the attachment wires 12 from an expanded configuration in FIG. 7 to a contracted configuration in FIG. 9, or for any other suitable purpose. The subsequent device 17 may be attached to the support wire 11 and/or the bulb 27 by any suitable mechanism for attachment, such as, but not limited to, a snap-on mechanism, magnet, male/female type connector, screw-in attachment, or twist-lock mechanism. Further, the wire 11 with the subsequent device 17 may be disassembled, as desired, by any suitable release mechanism, such as, but not limited to, a switch, lever, heat, magnet, current applied, or current located to a control handle.

In addition, FIG. 10 depicts in another embodiment a mechanism to separate the attachment wires 12 from the prosthetic graft 1. An electrical current is applied to insulated 68 wires 11. In an embodiment, a high resistance connection 18 may then separate the attachment wires 12 from a thin wire or suture 19 disposed at or near the graft 1. For example, the connector 18 may be a metal alloy that weakens/separates when subjected to a current or it may be a filament that heats and severs an adjacent suture or some other such connector element that allows separation when a current is applied. FIG. 10 depicts a bipolar approach, but a monopolar mechanism with the ground being an adjacent arterial wall is also considered within the scope of the present invention. Thus, it is intended that the present invention cover all such modifications and variations of the invention, provided they come within the scope of the appended claims and their equivalents.

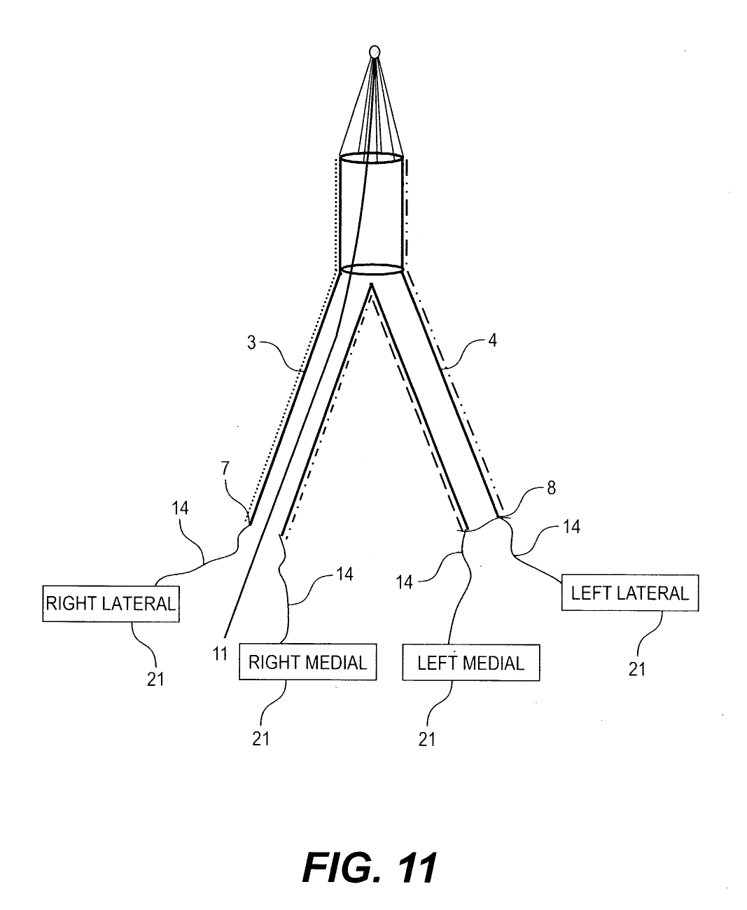

As shown in FIG. 11, a further embodiment includes a plurality of sutures 14 added to the end portions of the limbs 7 and 8. The sutures 14 may be attached to identification tags 21. The sutures 14 may also be removed when the limbs are trimmed to the appropriate length, at which time they may be replaced. The sutures 14 may be included in the packaged graft in order to provide a certainty as to the identification of the right 3 and left 4 limbs as well as the medial 9 and lateral 10 portions of the limb.

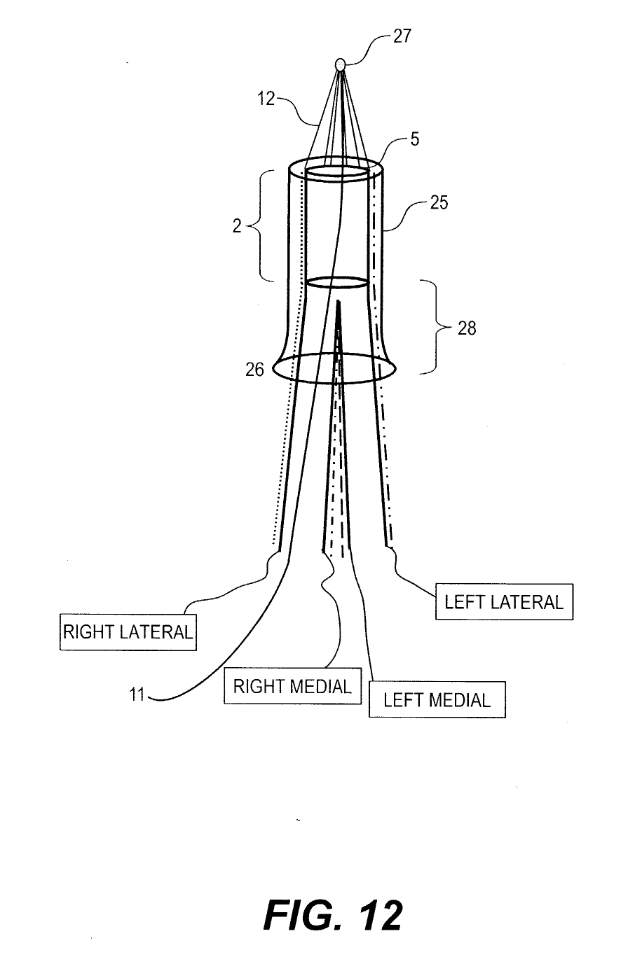

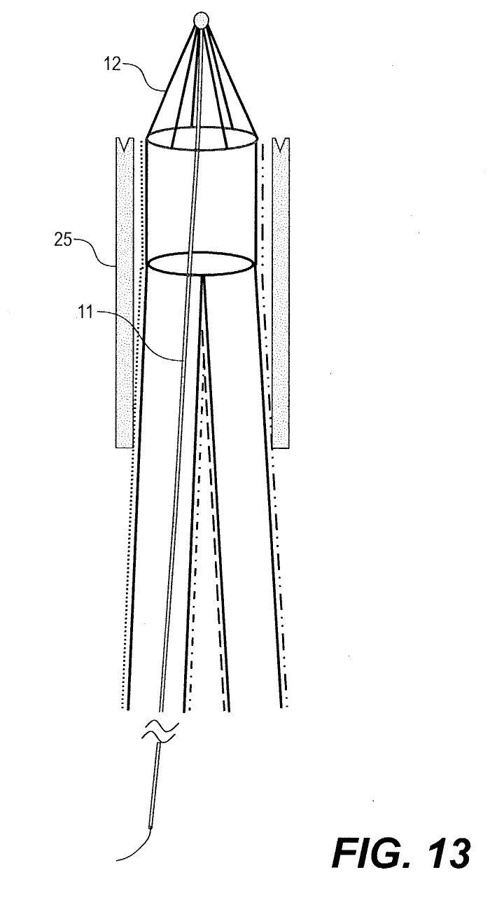

As depicted in FIGS. 12 and 13, the graft 1 may be packed in a short sheath 25. In an embodiment, the short sheath 25 may be flared at the bottom 26, as shown in FIG. 12. In FIGS. 11, 12, and 13, the support wire 11 may be attached to the attachment wires 12 at bulb 27. In FIG. 5, the attachment wires 12 may be attached to the filament balls 15 that, in turn, are attached to sutures 14 that, in turn, are attached to the top 5 of the tube portion of the graft 1. This arrangement allows for the bulk of the bulb 27 of the support wire 11, the attachment wires 12, the filament ball 15 in FIG. 5 and the knots 16 in FIG. 5 to be above the top of the graft as the graft is placed within one or more sheaths. The ball 15 is located at or near the tube portion 2 of the graft 1 whereas the bulb 27 is located above the tube portion 2 securing the attachment wires 12. The ball or bulb 15 or 27 can be the same structural element or may be different to accommodate the release mechanism. In an embodiment, the tube portion 2 may be a fixed length, such as, but is not limited to, about 2.5 cm and the upper portion of the limbs 28 included in the short sheath 25 may be, but is not limited to, about 3 cm. The tube portion 2 and the upper portion of the limbs 28 included in the short sheath 25 may be any suitable size depending upon the surgical procedure.

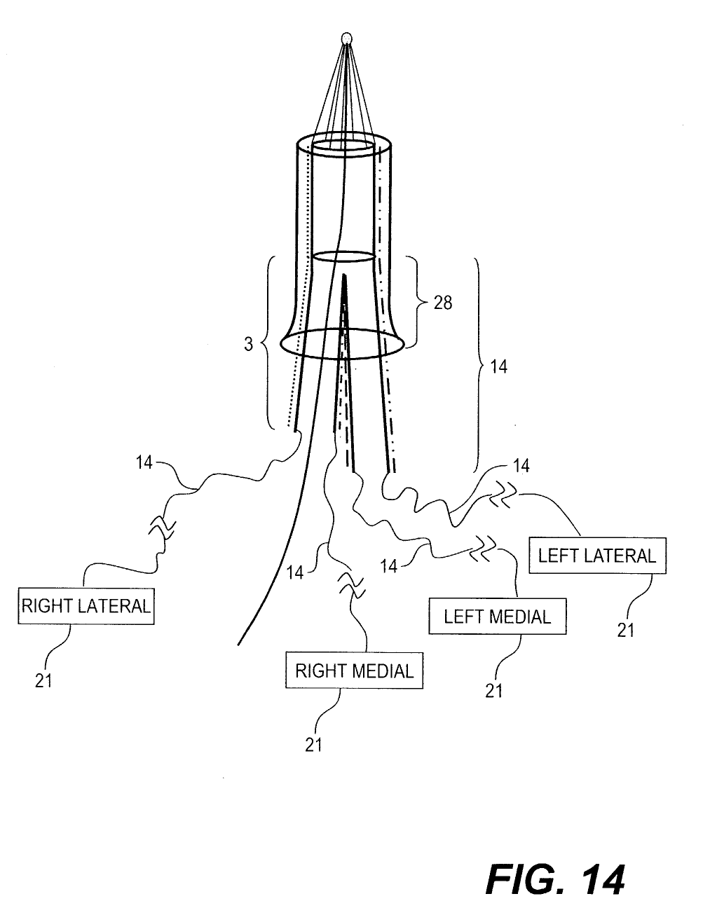

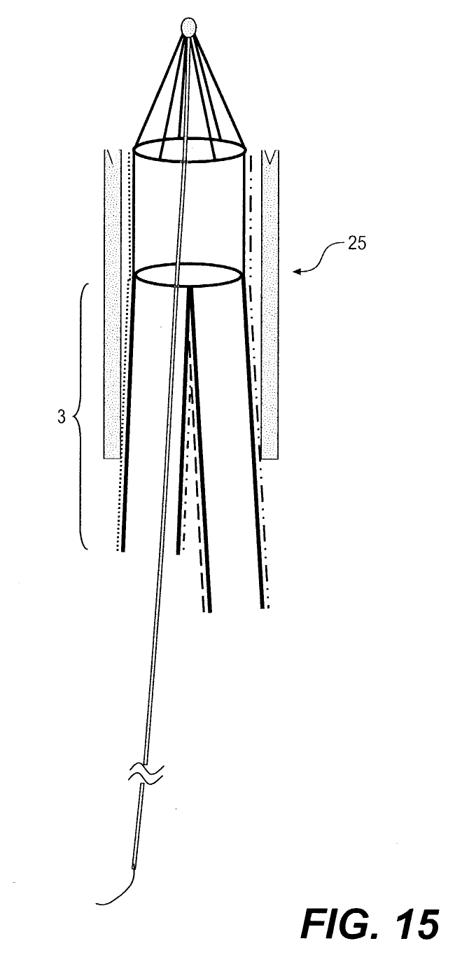

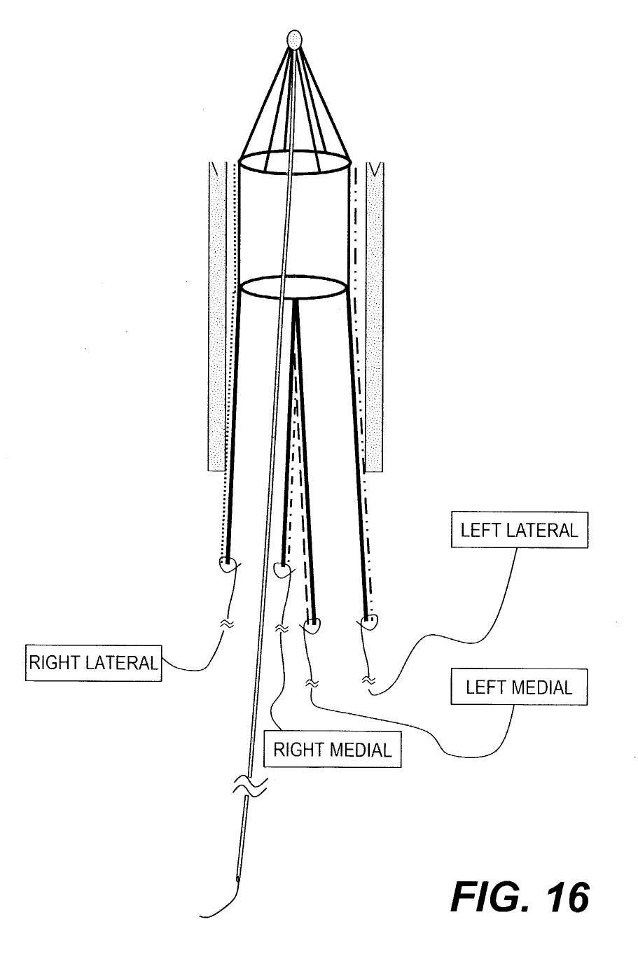

In another embodiment, shown in FIGS. 14 and 15, once the surgical component package is opened by the inserter, the right limb 3 may be cut to the appropriate length, as determined by measurements made from a suitable imaging modality, including, but not limited to, a 3D CT scan. The left limb 4 may be trimmed also. In FIGS. 14 and 16, longer sutures 14 are attached to the graft limbs 3 and 4, and the manufacturer supplied identification tags 21 may be applied or reapplied to the ends of the sutures.

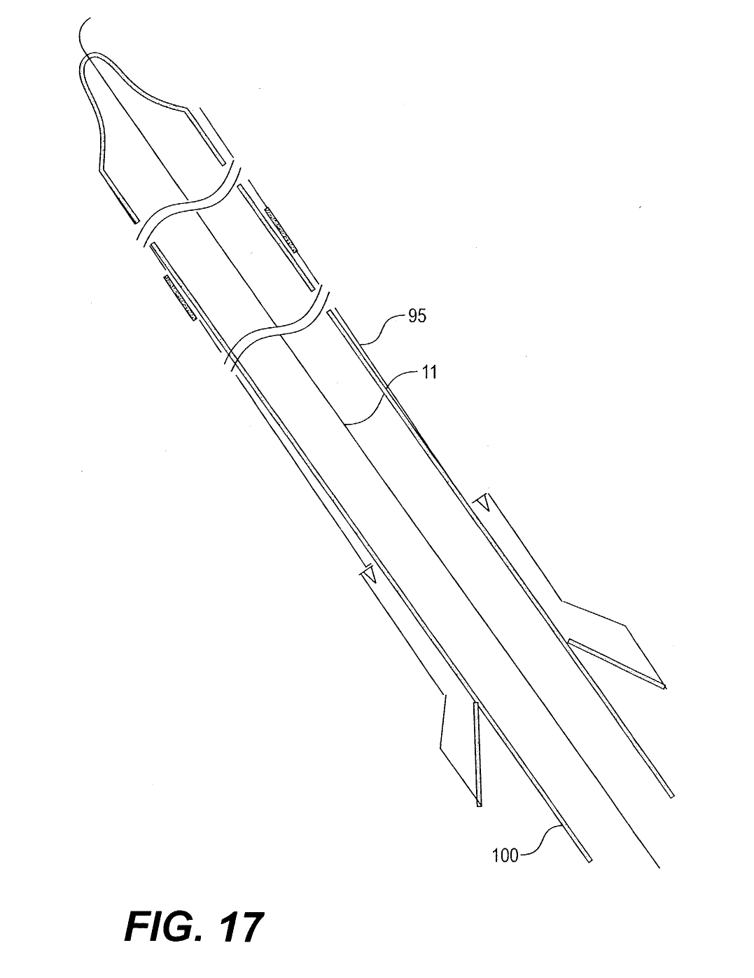

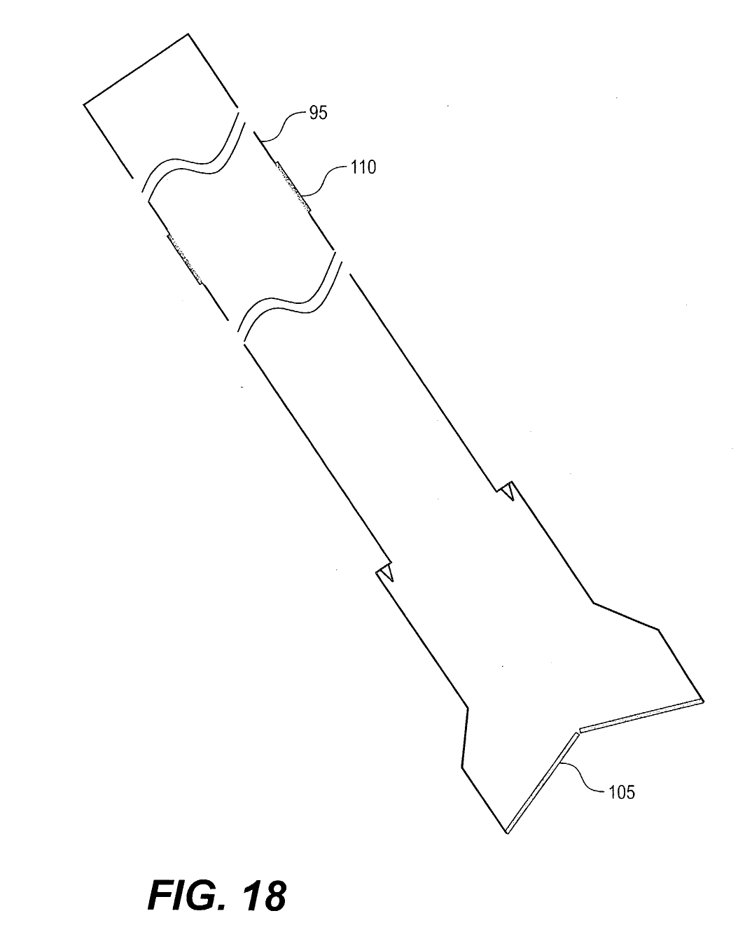

In another embodiment shown in FIG. 17, an introducer sheath 95 with a detachable hemostatic valve 105 and a dilator 100 are illustrated as another embodiment of the present invention. The introducer sheath 95 and dilator 100 may be passed over a support wire 11 from the right or left femoral artery up to the aortic neck. In FIG. 18, the introducer sheath 95 may have a detachable or non-detachable hemostatic valve 105, and the dilator 100 and wire 11 may be removed once the end of the introducer sheath 95 is placed at the level of the aortic neck. The introducer sheath 95 may have a soft or resilient portion 110 that can be clamped off.

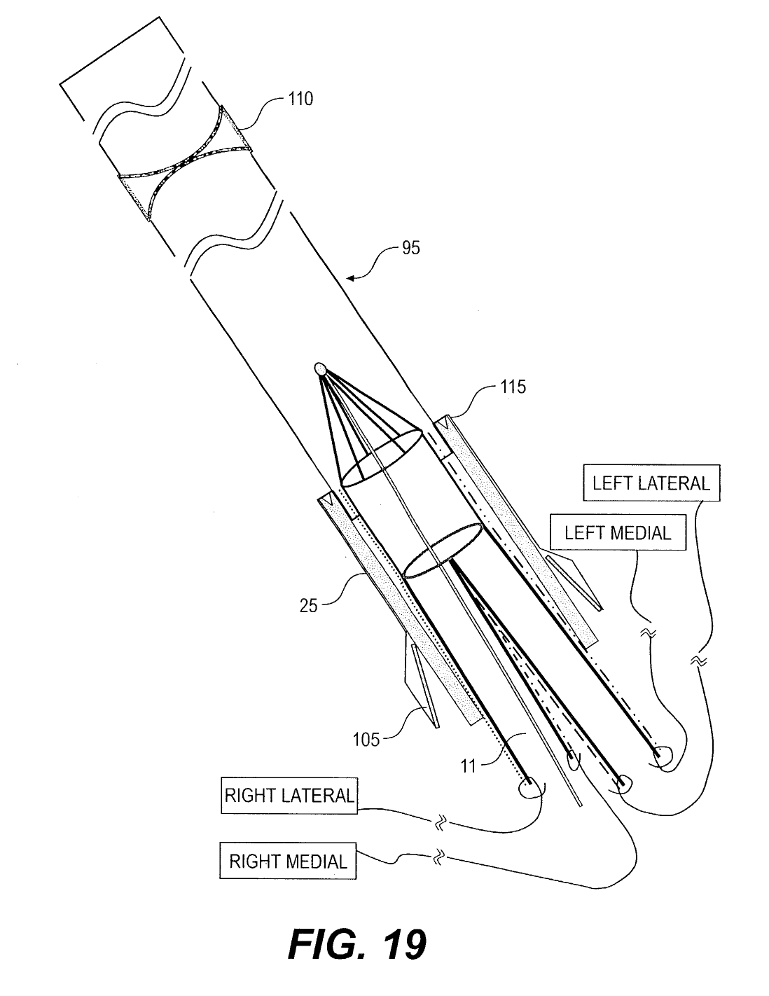

As shown in FIG. 19, the short sheath 25 containing the graft may be introduced into the introducer sheath 95 and advanced until it meets a docking point 115. The lumen of the short sheath 25 and that of the introducer sheath 95 may now be aligned so that easy transfer of the graft from the short sheath 25. The introducer sheath 95 is stationary while the support wire 11 advances the graft within the lumen of the introducer sheath. As the insertion apparatus is being introduced into the introducer sheath 95 the soft or resilient area 110 may be occluded to prevent blood loss while the hemostatic valve 105 is held open.

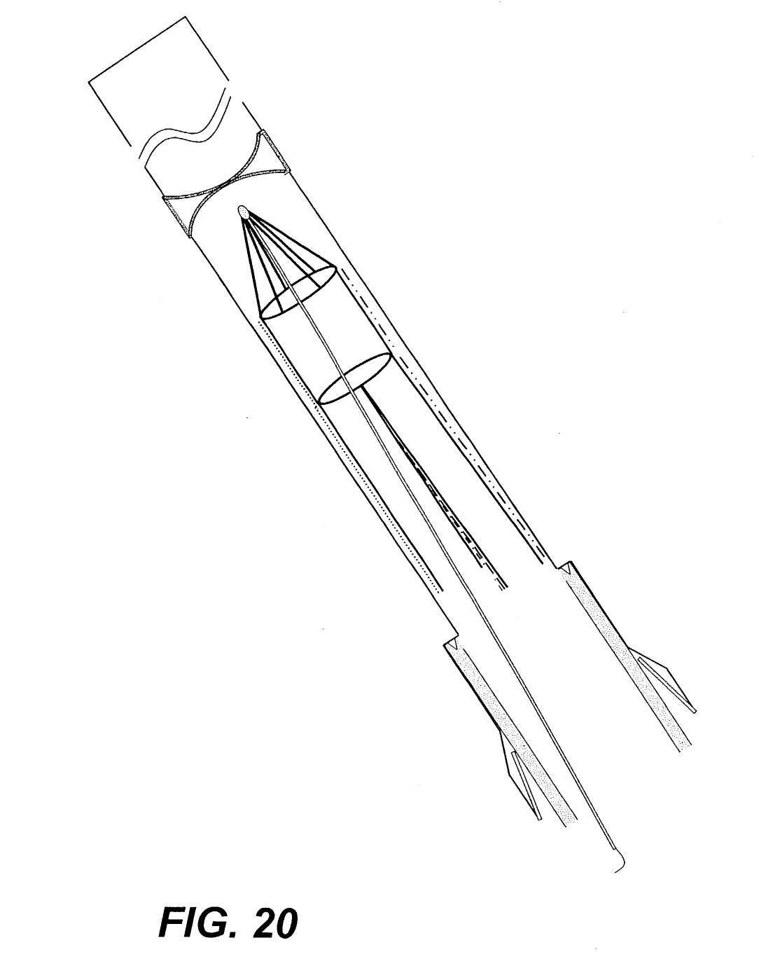

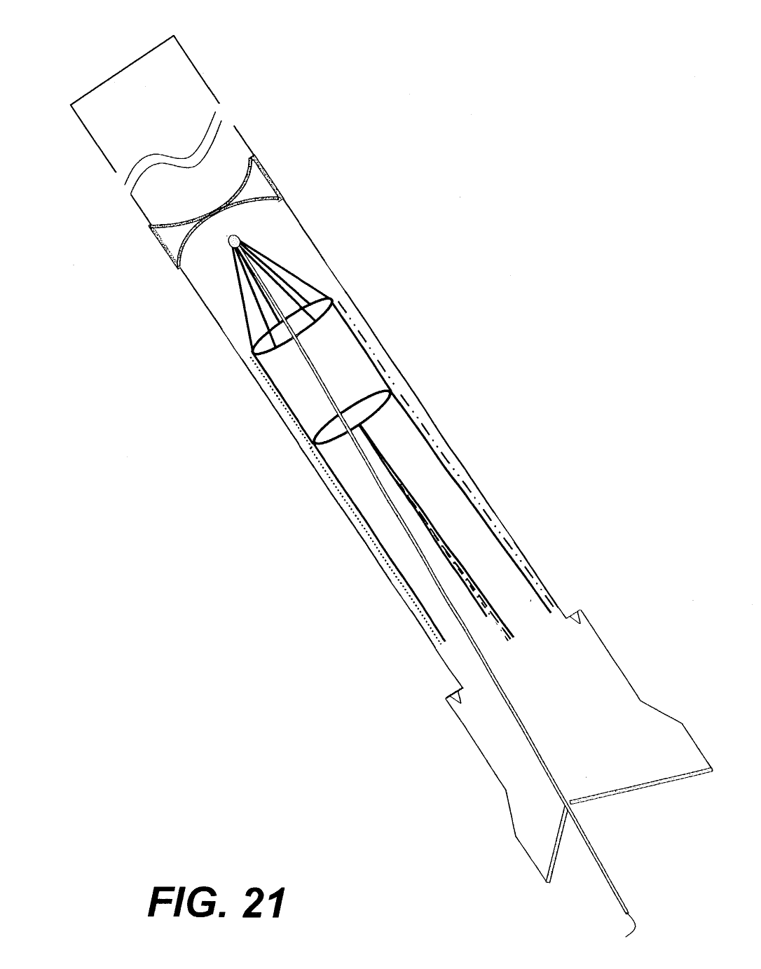

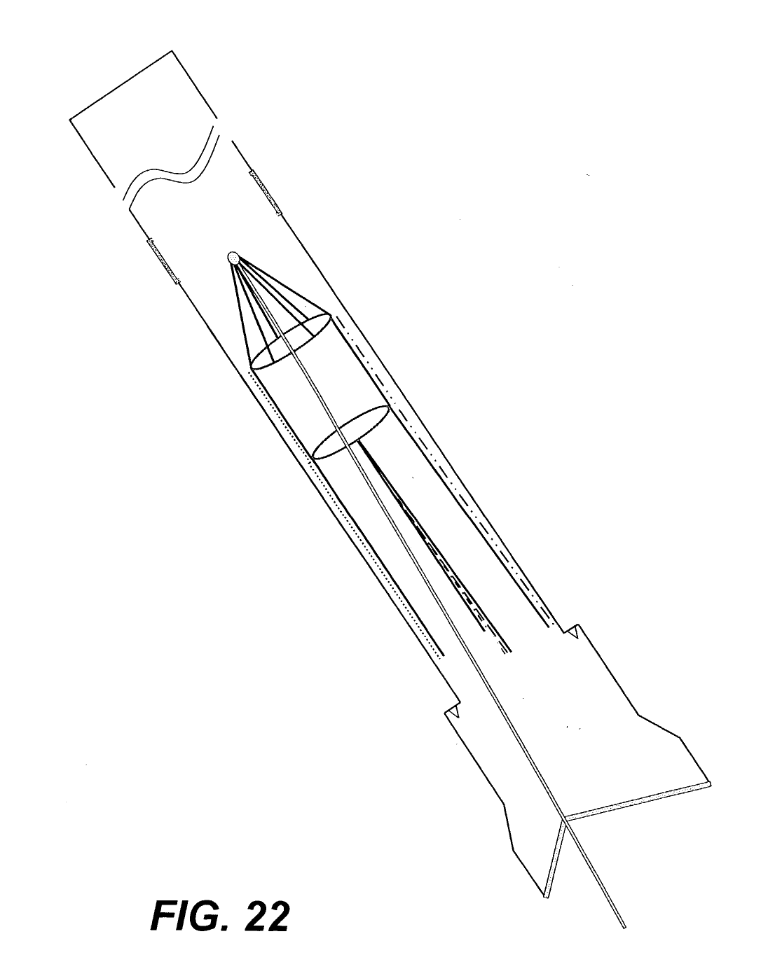

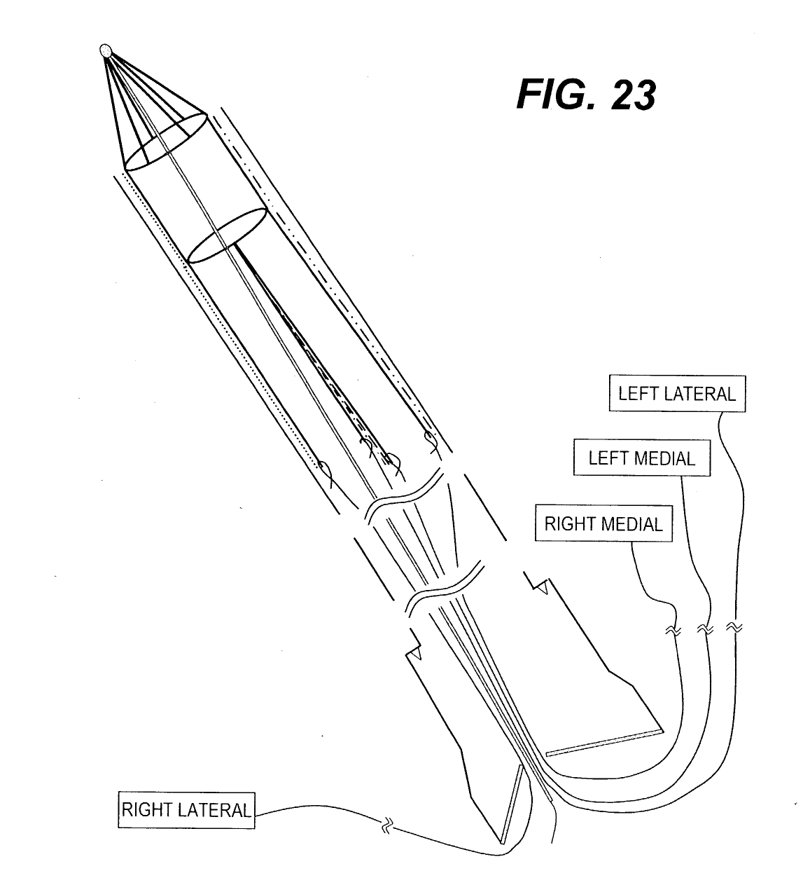

In FIG. 20, by pushing on the support wire 11, the graft may be advanced to the area just before the soft area 110 of the introducer sheath 95. At this point, the graft may be entirely removed from the short sheath 25. In FIG. 21, the short sheath 25 is removed. In FIG. 22, the occlusion mechanism at the soft or resilient area 110 is removed. The graft may be advanced to the end of the introducer sheath 95 and poised for release. FIG. 23 depicts the attachment wires 12 in a spread-out manner when in fact the wires may be tightly compressed against the support wire and may only be released once the restraint of the introducer sheath is removed.

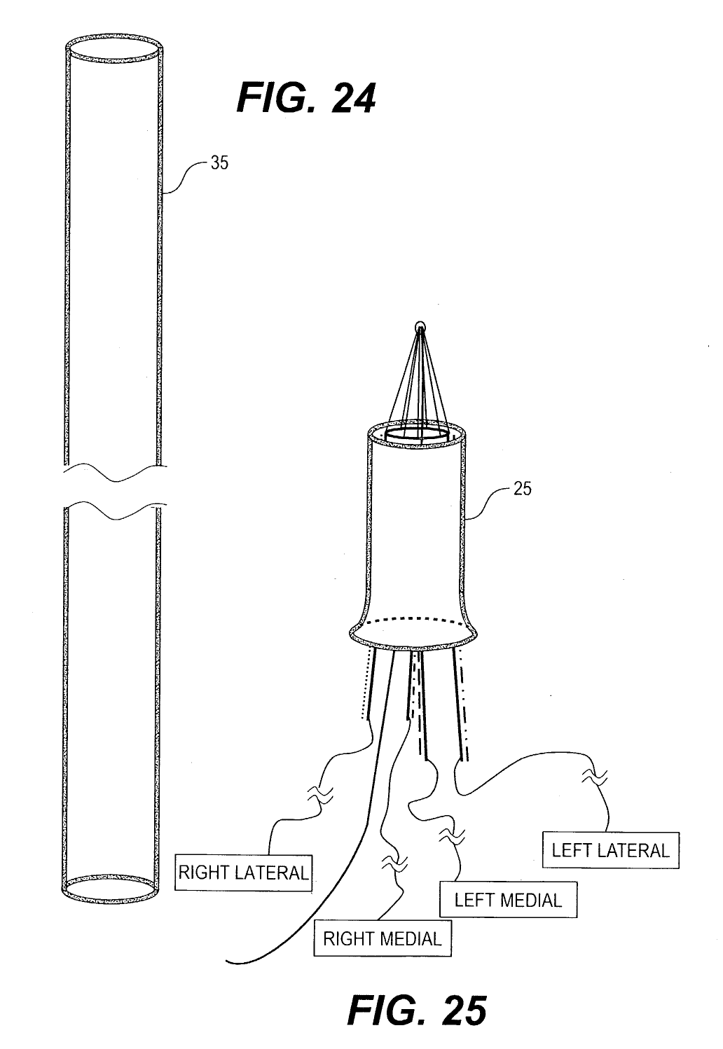

According to another embodiment of the present invention, FIG. 25 depicts a surgical component, which may be supplied to a physician in a short sheath 25 (now shown as opaque). A long delivery catheter 35, as depicted in FIG. 24, may be brought into the sterile field. The long delivery catheter 35 is capable of receiving the contents of the short sheath 25 once the graft limbs are trimmed to the appropriate length and long sutures, with attached tags are reattached to the graft limbs. The long delivery catheter 35 is also capable of insertion into the introducer sheath 95. Thus, the long delivery catheter 35 must be of appropriate length and diameter to facilitate insertion within the introducer sheath to reach the vessel for repair.

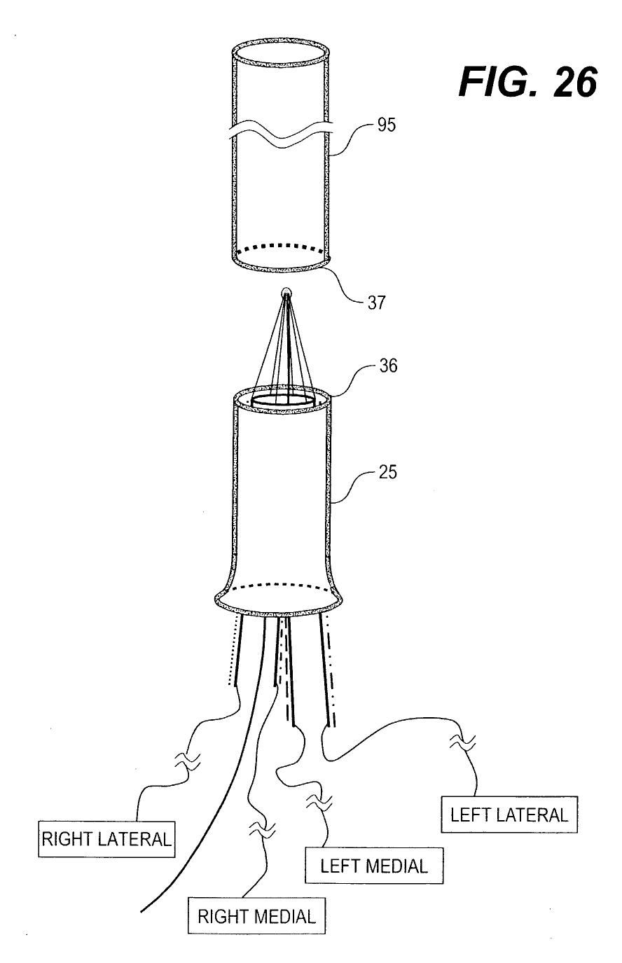

According to FIG. 26, at the end 37 of the long delivery catheter 35 opposite the portion that will be inserted into an artery there may be a connection mechanism so that a port or multiple ports with hemostatic seals can be attached. This will allow the support wire, the sutures attached to the graft limbs, and any other device to be inserted into the patient to either share a hemostatic port or to have separate ports for each or some combination of the above.

As shown in FIG. 26, the top 36 of the short sheath 25 is placed at the end 37 of the long delivery catheter 35 in preparation for the transfer of the graft assembly from the top 36 the short sheath 25 into the long delivery catheter 35.

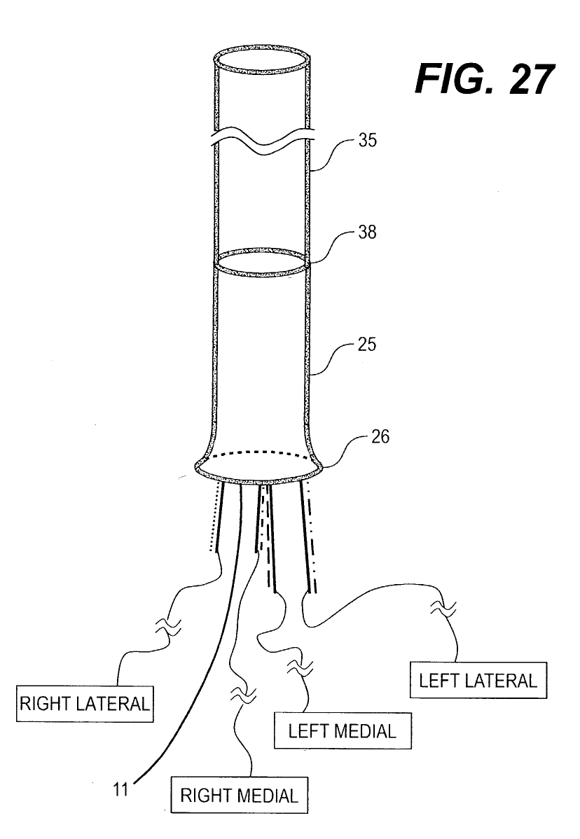

As depicted in FIG. 27, the short sheath 25 may be joined at its top to the long delivery catheter 35 at its bottom to form a junction 38. The junction 38 may be a locking joint that makes the inside appear seamless. The flare on the short sheath 26 assists in helping feed the graft into the short sheath 25 as the graft is pushed into the long delivery catheter 35 by pushing on the support wire 11.

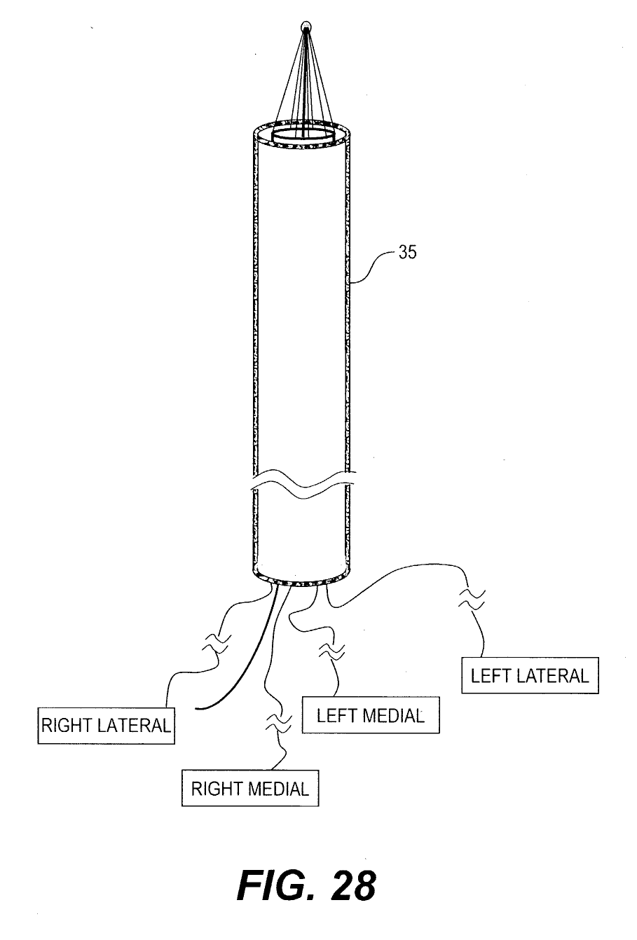

In FIG. 28, the entire graft has been advanced into the long delivery catheter 35 and the short sheath 25 has been removed. This graft transfer may be expedited by having a lubricious inner surface on the sheaths.

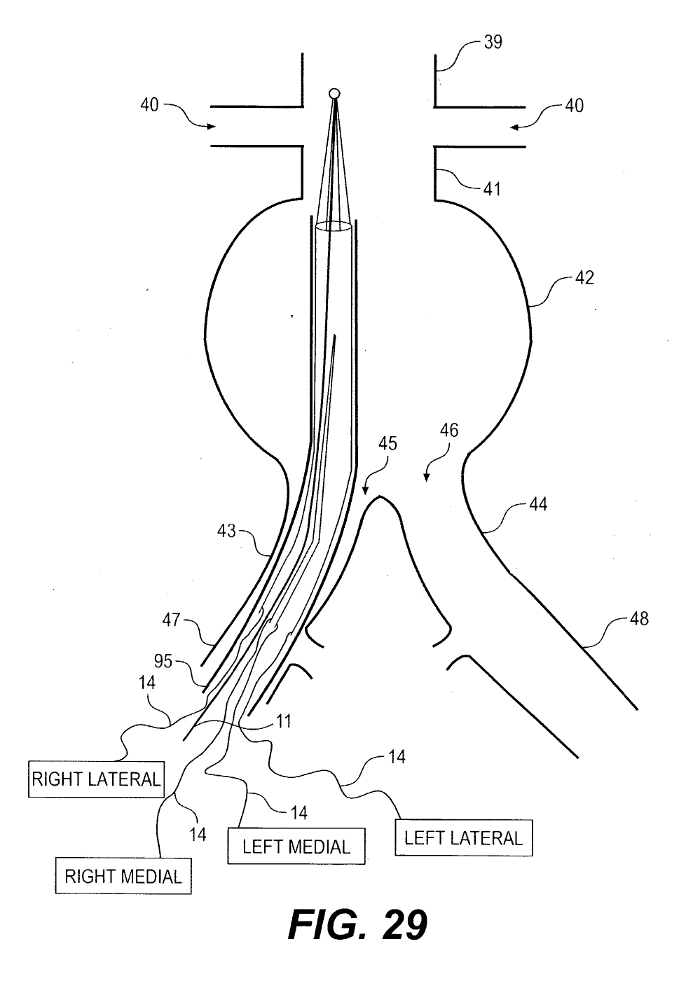

In FIG. 29, the aneurysm diagram depicts a suprarenal aortic portion 39, renal arteries 40, an infrarenal aortic neck 41, an abdominal aortic aneurysm 42, right 43 and left 44 common iliac arteries as well as their orifices 45 and 46, and right 47 and left 48 external iliac and common femoral arteries. According to an embodiment of the present invention, the introducer sheath 95 may already have been inserted into the right femoral artery and advanced up to the level of the aortic neck 41. The support wire 11 and the sutures 14 attached to the graft limbs both exit from the introducer sheath 95. In an alternate embodiment of the present invention, the long delivery sheath 35 is inserted into an introducer sheath (not shown) in the right femoral artery 47 and advanced up to the level of the aortic neck 41. The introducer sheath 95 inserted into the common femoral or external iliac artery 47 over a guide wire at the beginning of the procedure can be left in place for much, though not necessarily all of the procedure.

The end portion of the introducer sheath that is positioned within the aortic neck may be manufactured to be straight when the dilator 100 (FIG. 17) is in place. It may remain straight or it may assume a curved configuration once the dilator 100 is removed. The ability to tip deflect the end of the introducer sheath 95 by controls at the opposite end of the introducer sheath 95 outside the body may be adopted as part of the manufacturing process of the introducer sheath 95. These controls may include manipulating wires through wires passing through longitudinal holes or by means of applying a current or some other force that would change the durometer of the end of the introducer sheath in a manner that would facilitate tip deflection. Such tip deflection would facilitate manipulating the tube portion of the graft 2 into an angulated aortic neck 41.

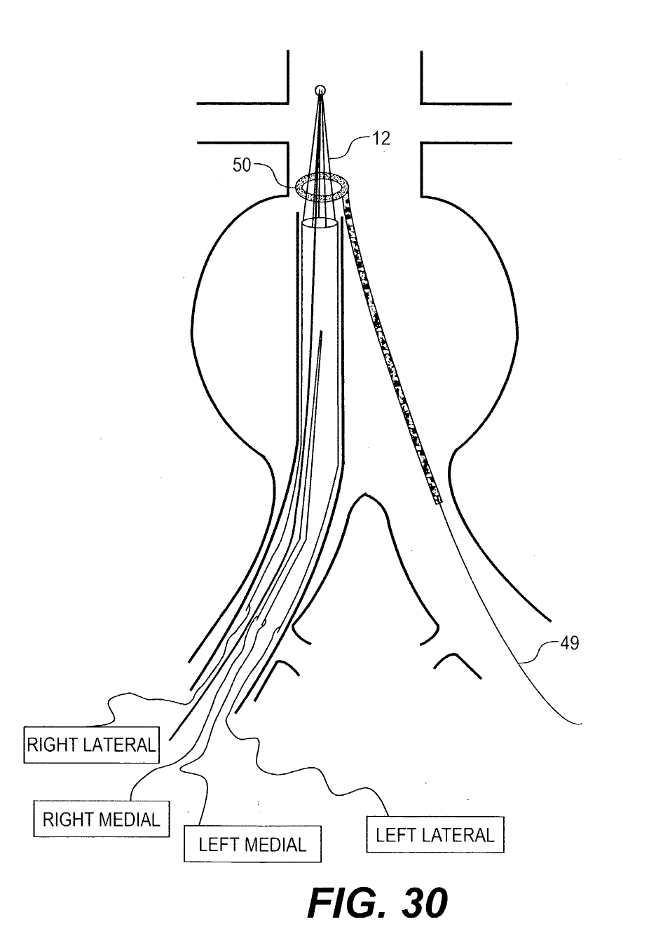

In an embodiment shown in FIG. 30, a snare catheter 49 may be inserted through the opposite femoral artery, in this example, the left femoral artery 48 sheath (not shown) and the snare component 50 may be advanced over the attachment wires 12. Use of a snare may reduce the introducer sheath's outside diameter. If space is available within the right larger introducer sheath, there may be a multiplicity of ways, for example, as referenced in the description of FIG. 7, to build in an expansion/retraction system to handle the expansion and retraction of the attachment wires 12.

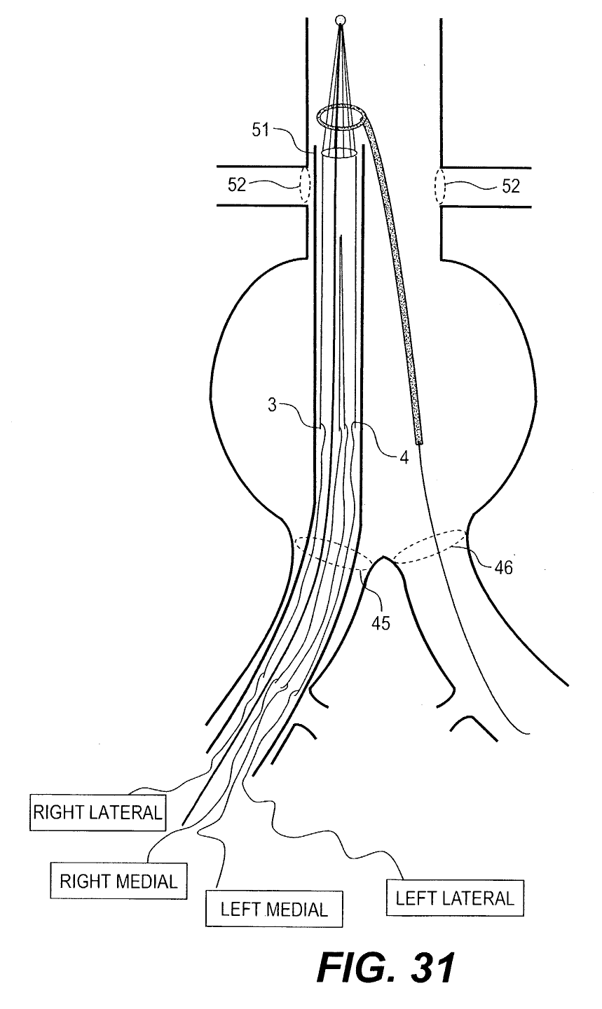

As shown in FIG. 31, the top of the introducer sheath 95 may be brought above the orifices 40 of the renal arteries so that the ends of the graft limbs 3 and 4 are above the orifices 45 and 46 of the common iliac arteries.

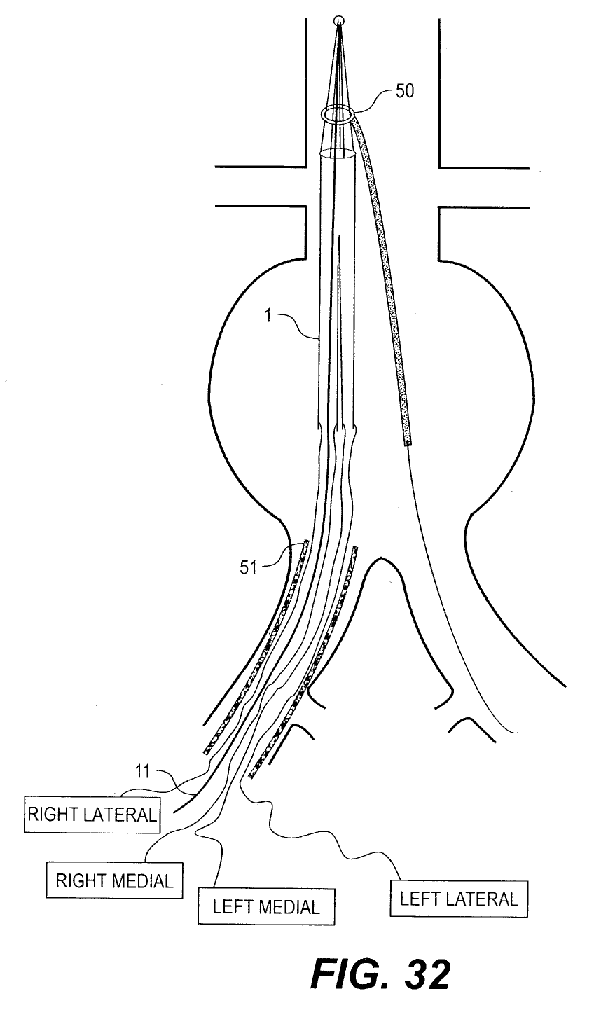

In FIG. 32, the top 51 of the introducer sheath 95 may be pulled back while the support wire 11 is held steady so that the entire graft 1 is released from the introducer sheath. In this embodiment the snare component 50 prevents the top of the graft from expanding. As referenced in the description of FIG. 7, a catheter could also be passed over the support wire 11 and connected to a mechanism at the bulb 27 that would allow control of the angulation of the attachment wires 12 which would make the use of the snare optional.

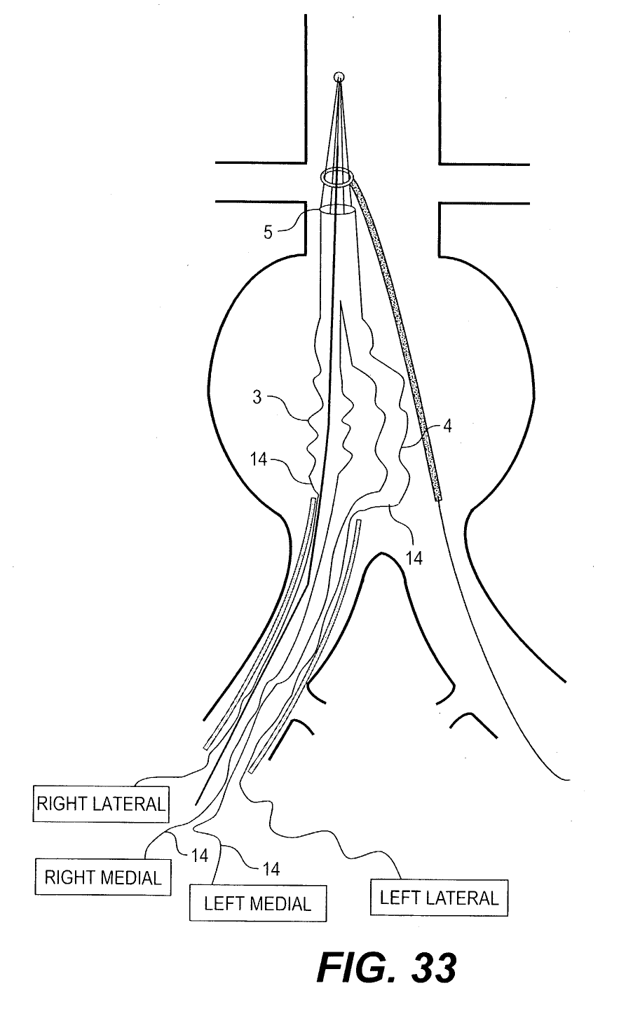

According to FIG. 33, the top of the graft 5 may be placed within the aortic neck. As the graft is released, the right 3 and left 4 limbs of the bifurcation graft may not be under tension from the attached sutures 14 and may be contained within the aneurysm.

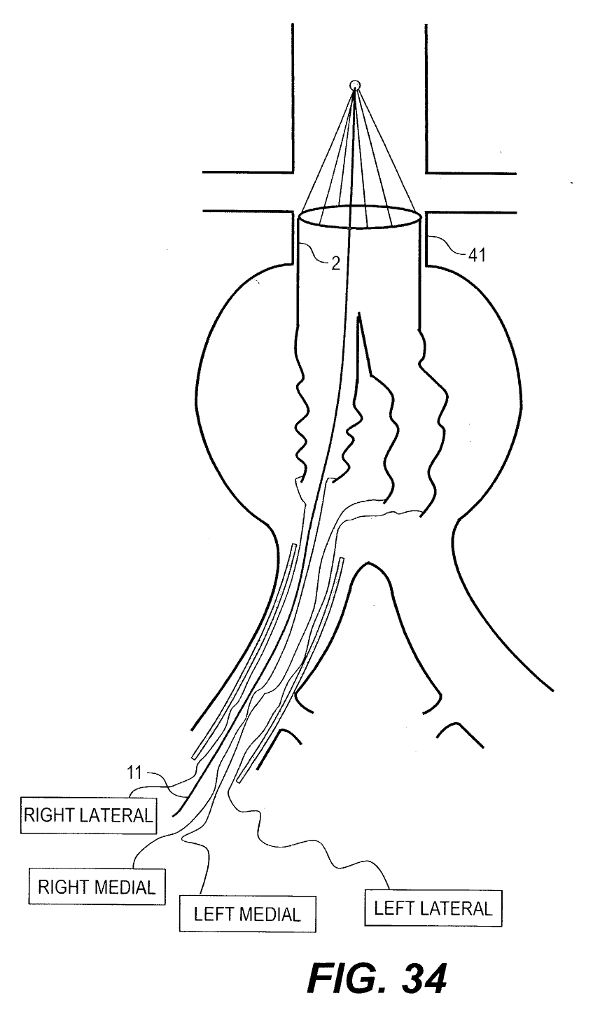

In FIG. 34, the snare component 50 is removed and, by holding the support wire 11 steady, the tube portion 2 of the graft remains in position within the aortic neck 41. The attachment wires 12, for the first time unrestrained, may hold the graft open. In an embodiment of the invention, the aorta is not occluded with the release of the graft. This approach may be less dangerous than techniques that require intermittent aortic occlusion because the heart is not subjected to large fluctuations in systemic vascular resistance. If the aorta is occluded near the level of the renal arteries, the pressure that the heart has to pump against is high. Indeed, systemic vascular resistance may be altered minimally with the approach presented in embodiments of the present invention.

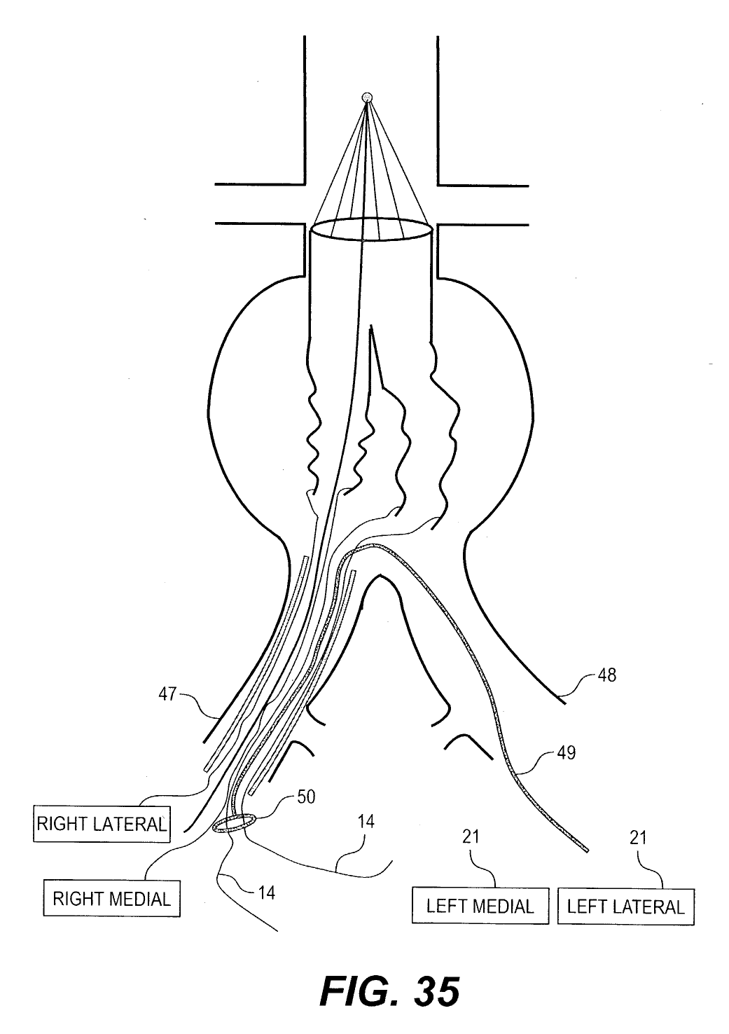

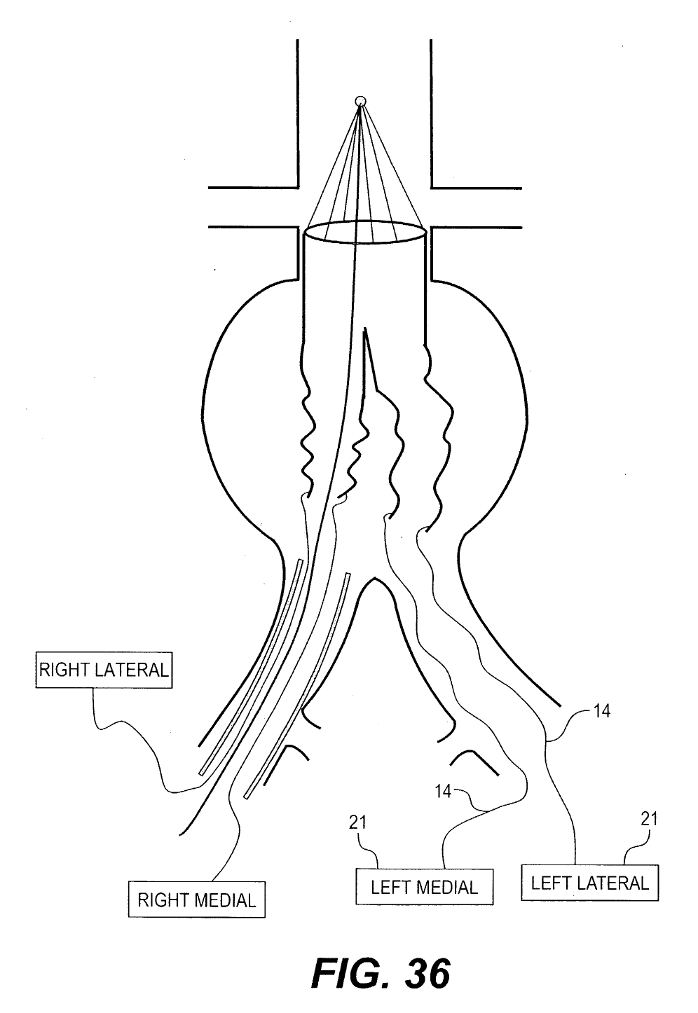

As shown in FIG. 35, in this example, a snare catheter 49 is passed from the left femoral artery 48 out the right femoral artery 47. The sutures 14 attached to the left limb 4 of the graft 1 may be placed within the snare component 50. In FIG. 36, the identification tags 21 for the left medial and left lateral have been detached and are reattached once the sutures have been passed to the left side.

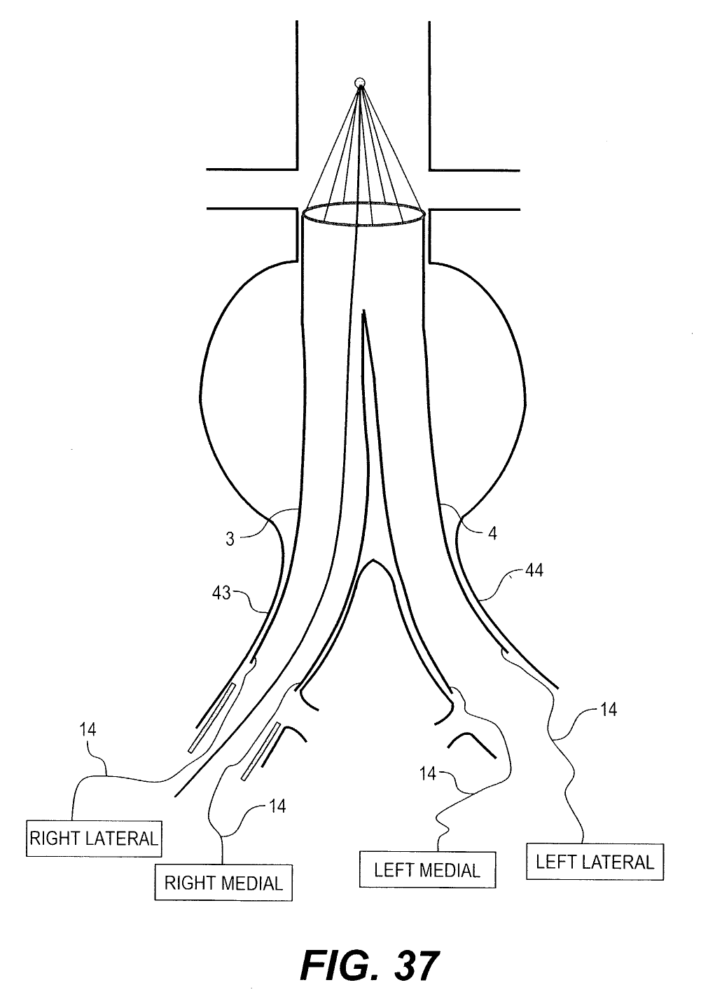

In FIG. 37, tension may be placed on the sutures 14 attached to the left limb 4 and also to the sutures 14 attached to the right limb 3 so that the limbs are brought down into the left 44 and right 43 common iliac arteries.

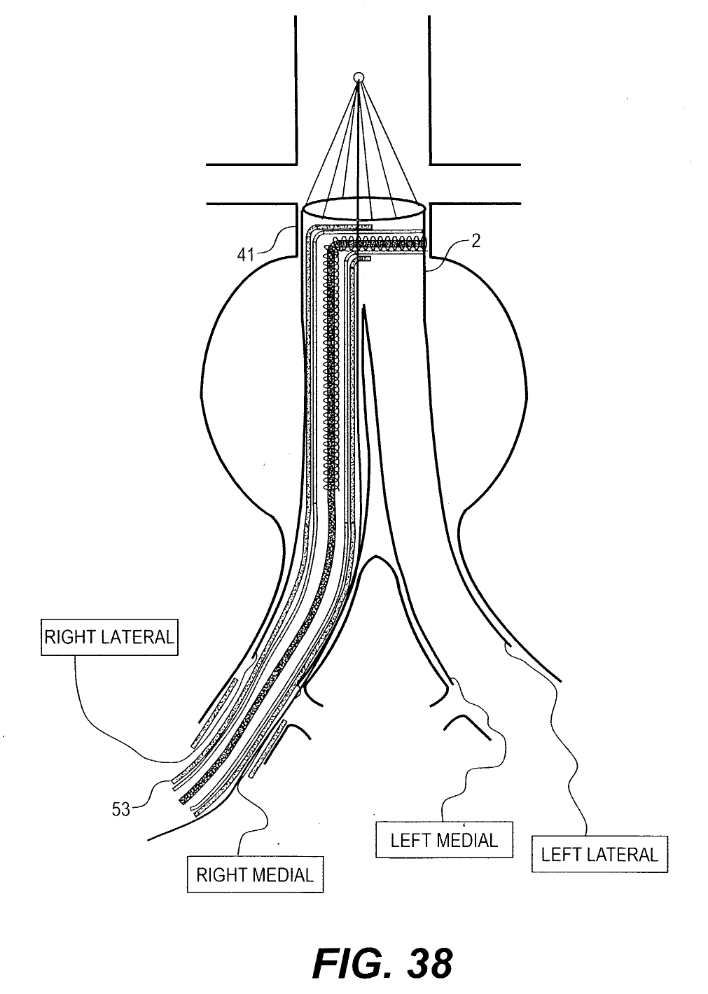

In an embodiment shown in FIG. 38, a fastener delivery catheter 53 may be inserted and positioned within the aortic neck 41 adjacent to or near the top of the tube portion 2 of the graft in order to initiate insertion of the fasteners 54. The fastener delivery catheter 53 disclosed in U.S. application Ser. No. 09/783,313 filed Feb. 15, 2001, the disclosure and the subject matter is hereby incorporated herein by reference. The fastener catheter 53 may also provide any suitable type of fasteners, including, but not limited to, metal alloy, plastic, suture, wire, or any other suitable fastening mechanism.

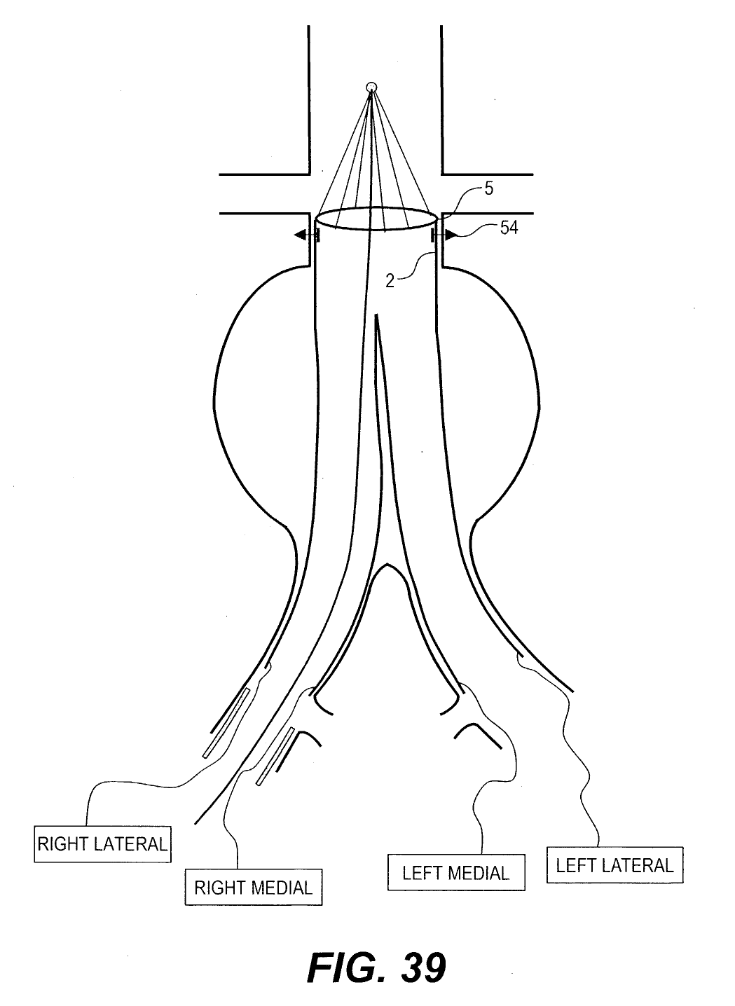

In an embodiment shown in FIG. 39, fasteners 54 may be inserted circumferentially around the top 5 of the tube portion 2 of the graft 1.

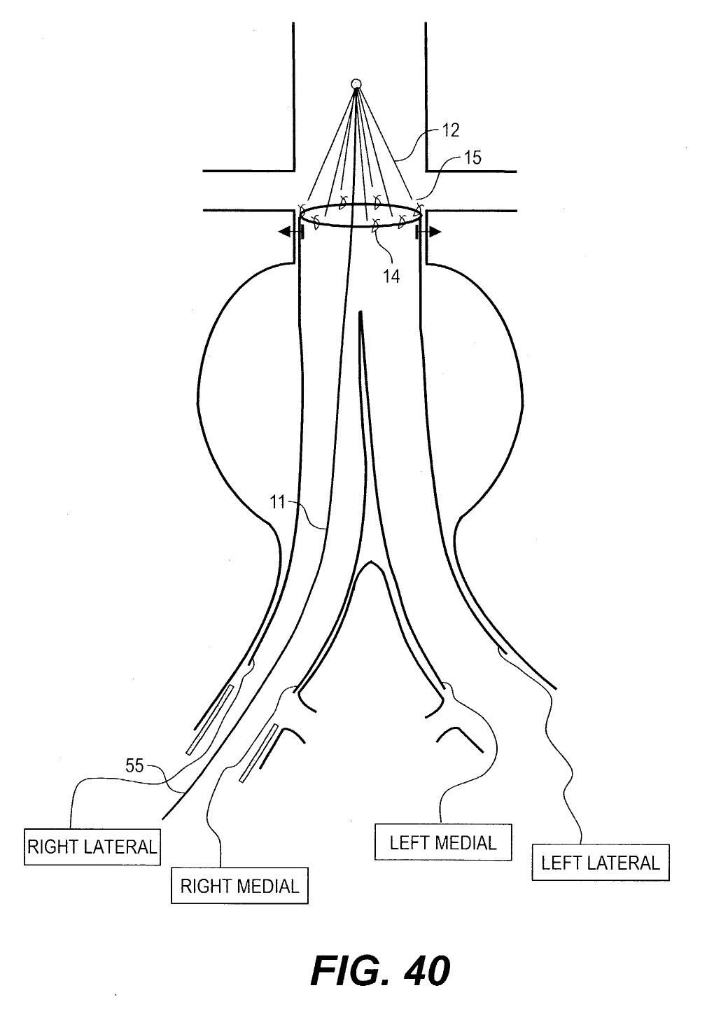

In an embodiment shown in FIG. 40, an electrical current may be applied to the support wire 11 by an actuator 55 outside the body of the patient. This causes the filaments attached to the attachment wires 12 to heat and sever the attached sutures 14. FIGS. 4, 5, and 6 detail an embodiment of the release mechanism using a current to increase temperature. Any suitable means may be used to effect release

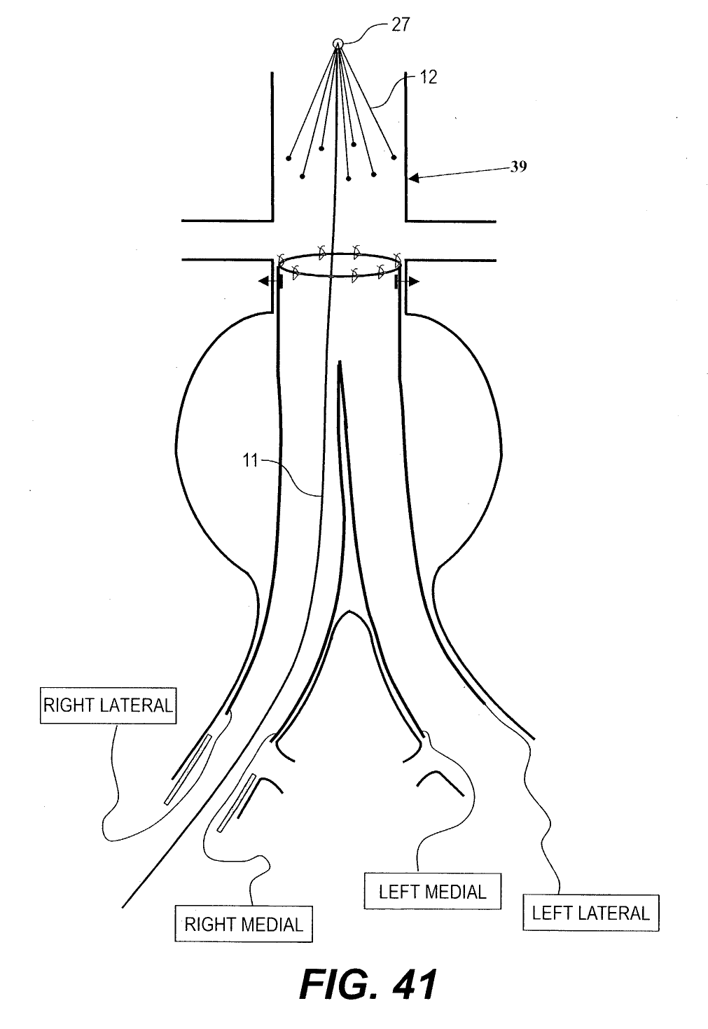

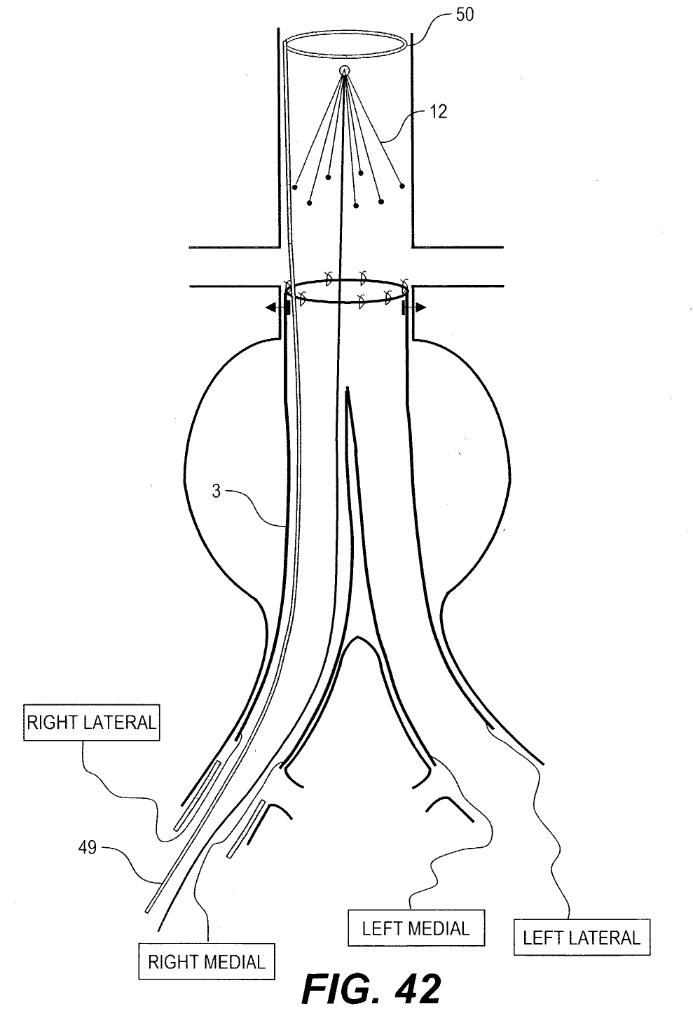

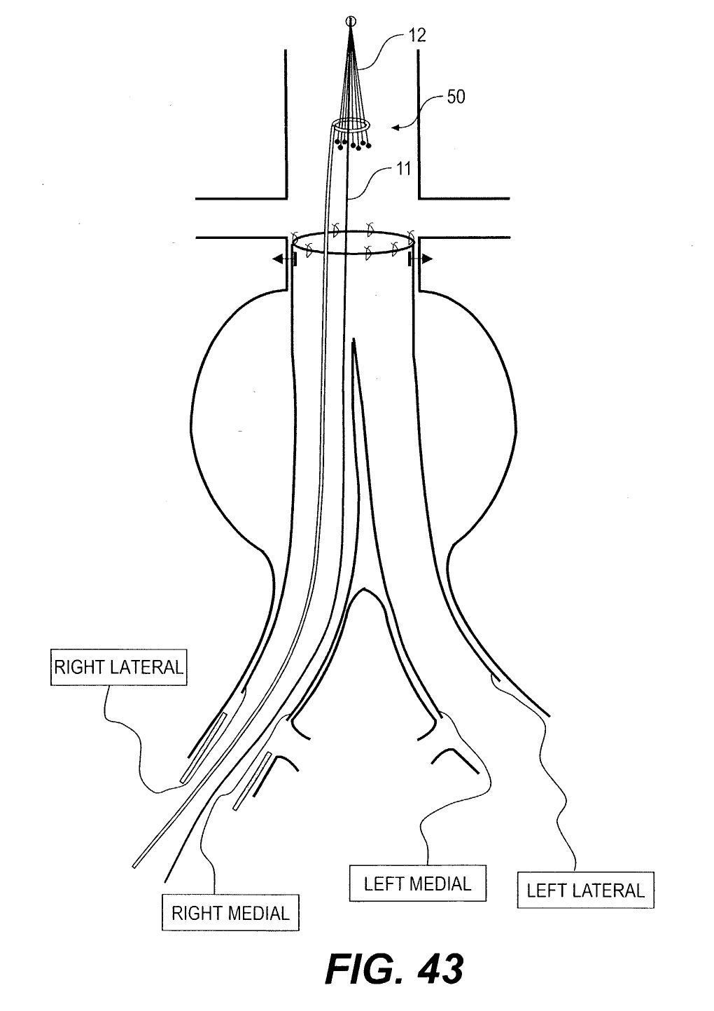

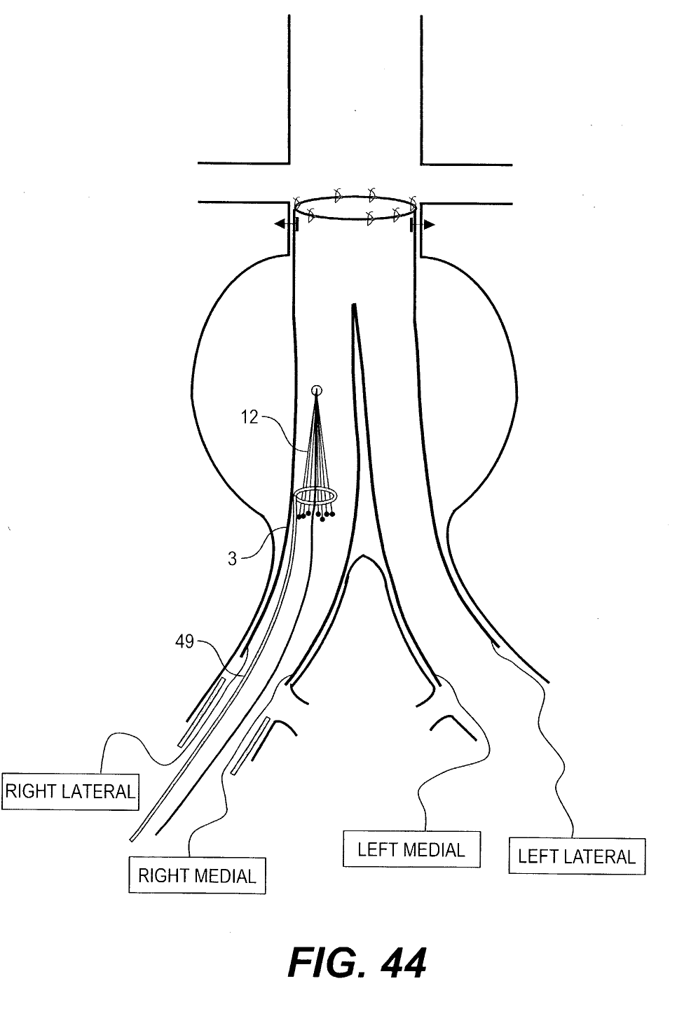

FIGS. 41 through 44 depict an embodiment of the release mechanism, and a method to remove the attachment wires 12 and the support wire 11 through the use of the snare component 50. In FIG. 41, by advancing the wire 11, the bulb 27, and the attached attachment wires 12 may be advanced into the suprarenal aorta 39. In FIG. 42, the snare catheter 49 is advanced through, by way of example, the right limb of the graft 3 and the snare component 50 is placed above the attachment wires 12. As shown in FIG. 43, the snare component 50 may be tightened around the attachment wires 12 so that they may be brought close to the support wire 11. In FIG. 44, the snare catheter 49 may be withdrawn through the right graft limb 3 bringing the attachment wires 12. As referenced in the description of FIG. 7, there can be multiple other methods of facilitating the transition between an expanded and a contracted configuration of the attachment wires.

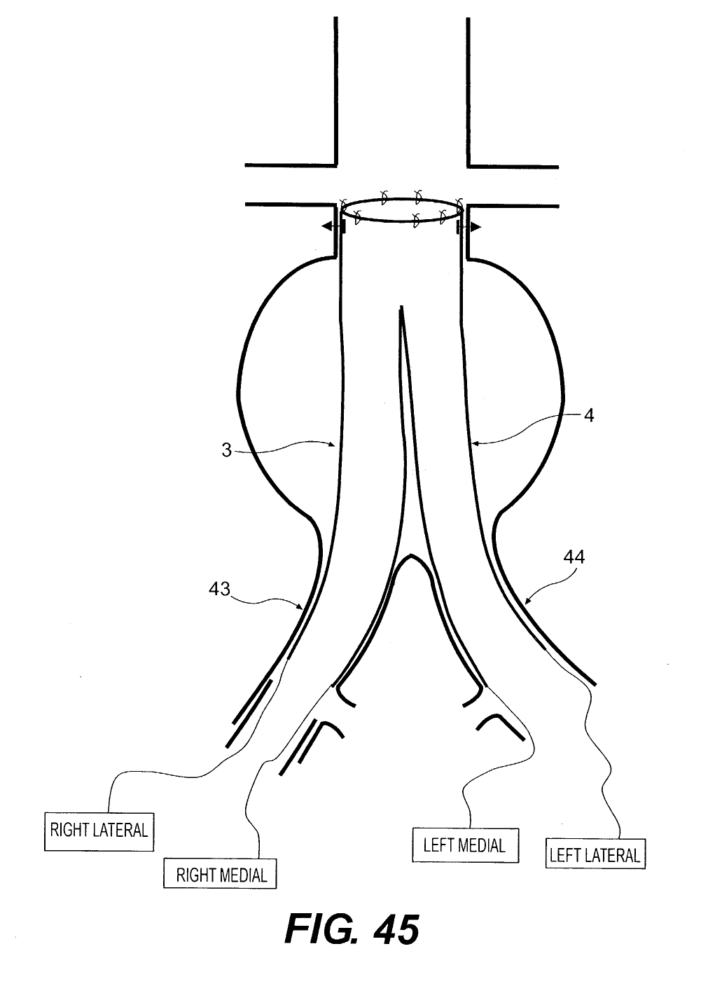

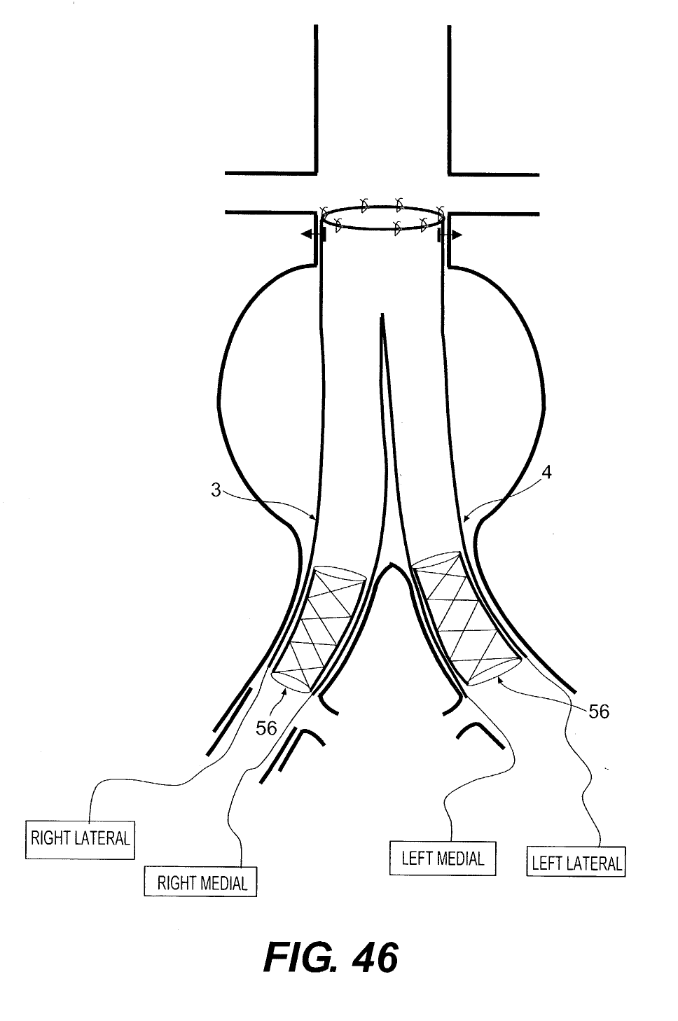

FIGS. 45-47 illustrate securing the graft limbs to the left 44 and right 43 common iliac arteries. As shown in FIG. 45, the attachment wires have been removed and the right 3 and left 4 graft limbs are now ready for attachment within the right 43 and left 44 common iliac arteries. In FIG. 46, two stents 56 have been inserted into the distal graft limbs 3 and 4 to provide an attachment means to the distal graft limbs.

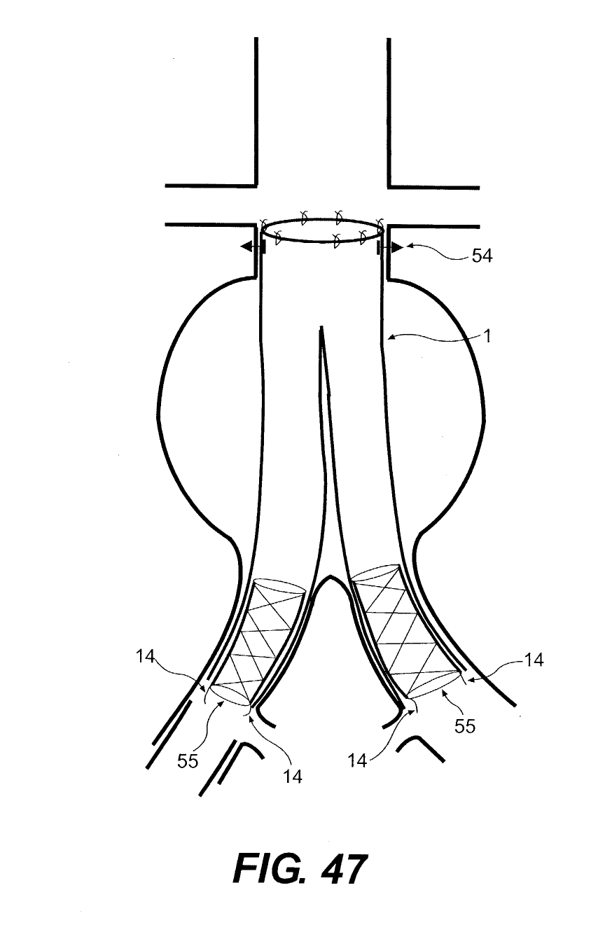

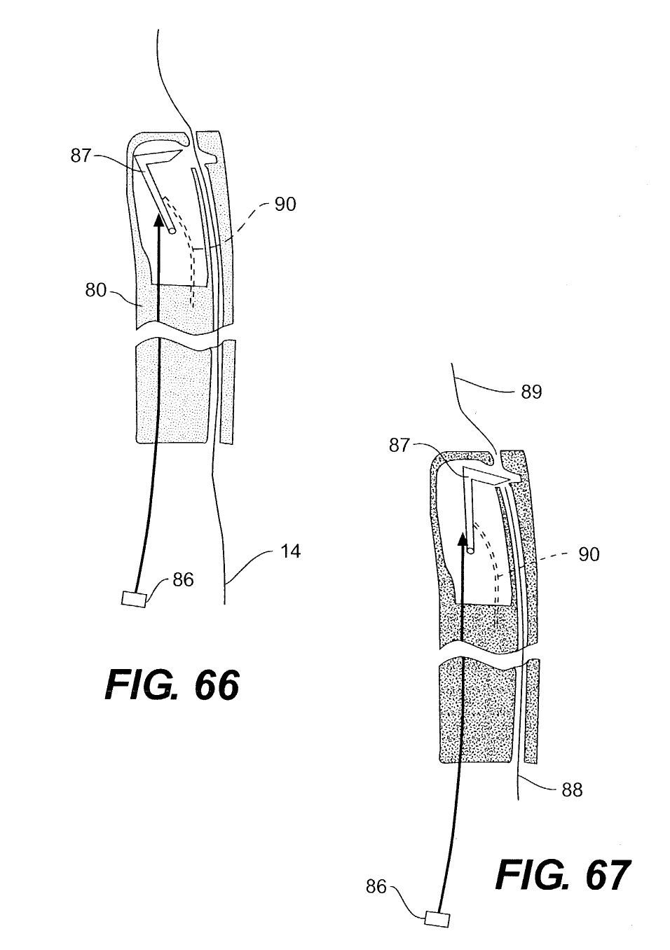

In FIG. 47, the sutures 14 have been cut with a suture cutter 80. The suture cutter 80 is shown in FIGS. 66 and 67, and described in more detail below. The graft 1 is now securely attached to the aortic neck with fasteners 54 and to the common iliac arteries with stents 55.

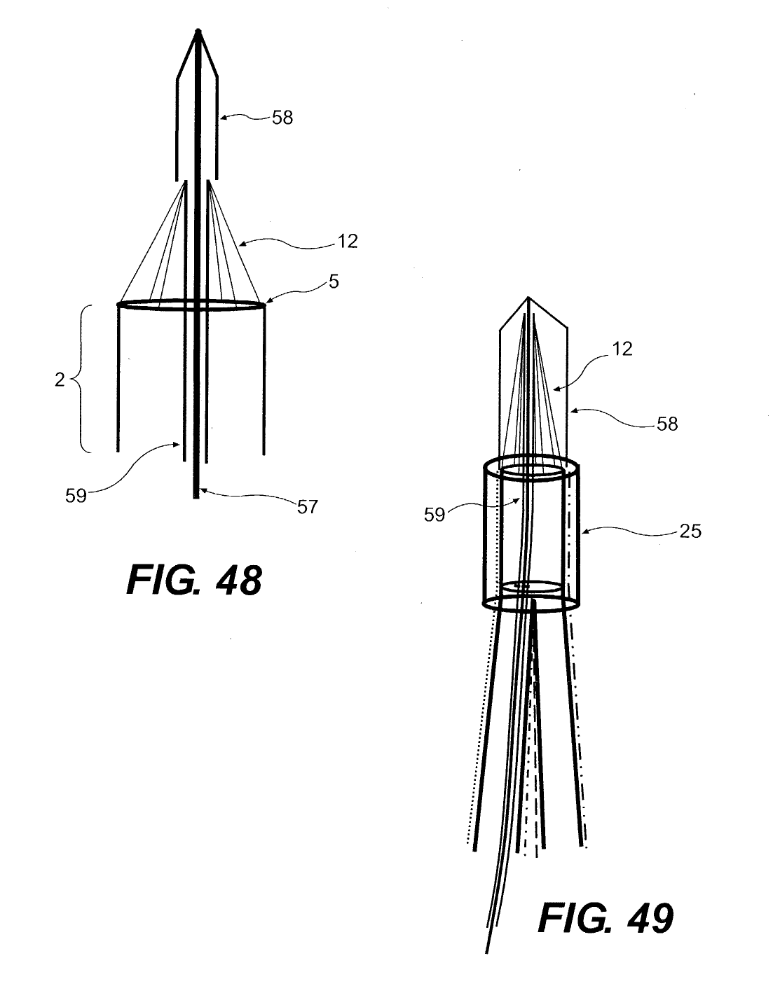

Instead of using the snare technique as shown in FIGS. 30-33 and FIGS. 42-44, an alternative embodiment uses a sheath as depicted in FIGS. 48, 49, and 54. The attachment wires 12 are attached to the top 5 of the tube portion 2 of the graft 1. The graft may be a supported graft or an unsupported graft. The insertion mechanism is composed of a central wire 57. The central wire 57 may be bendable by the inserter to facilitate inserting the graft into an angled aortic neck. The central wire 57 attaches to a containment sheath 58 above the top 5 of the tube portion 2. Instead of the attachment wires 12 being attached to the support wire 11, they now are attached to a catheter 59 surrounding the central wire 57. The central wire 57 and the support wire 11 function in a similar manner and may be made of the same material. FIG. 48 depicts the attachment wires 12 unsheathed.

The embodiment shown in FIG. 49 illustrates a way of supplying the graft positioned within a very short sheath 25 similar to the method described in FIG. 12. In this instance, however, the attachment wires 12 are enclosed and compressed toward the catheter 59 by the containment sheath 58. Note that the manufacturer can supply the graft with just the tube portion in a short sheath 25. The short sheath 25 may extend from the tube portion 2 to a portion over the limbs 28, as depicted in FIG. 12 or may extend just over the tube 2, as depicted in FIG. 49.

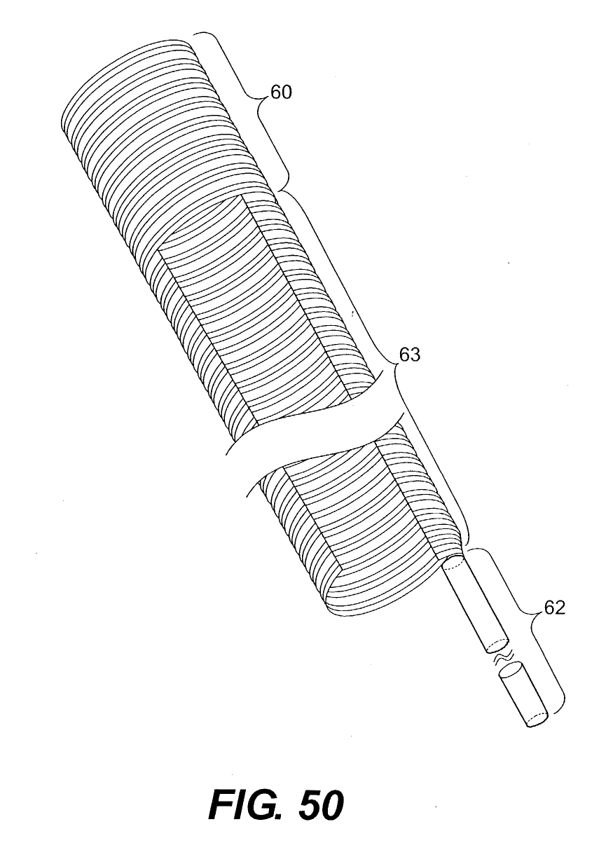

FIGS. 50, 51, and 52 provide additional embodiments of the present invention.

FIG. 50 provides a depiction of a very short sheath 25 connected to an open or partial sheath 63. If desired, this can be connected to a solid component such as, but not limited to, wire or a synthetic, such as plastic or an open catheter 62.

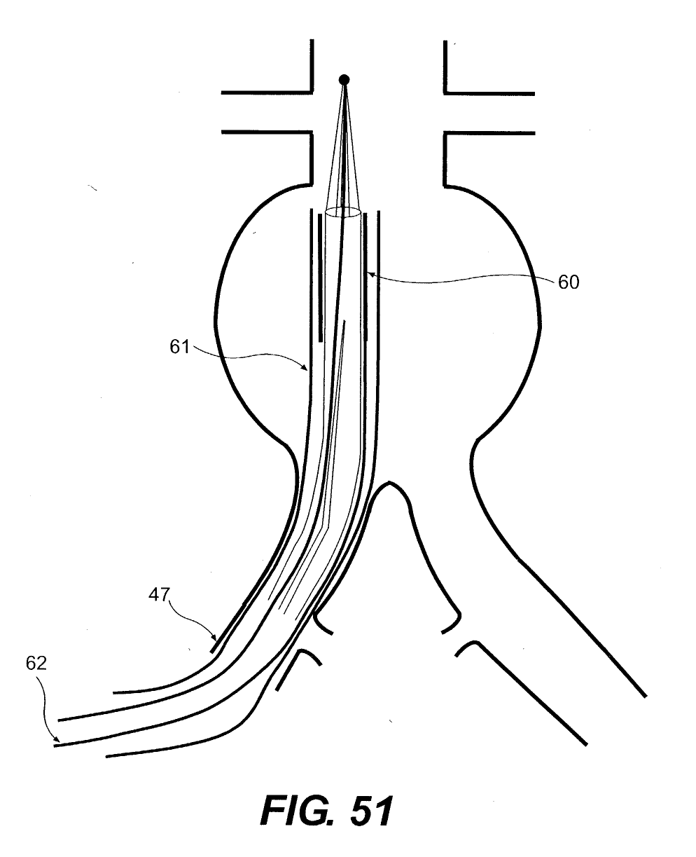

FIG. 51 depicts such a sheath in position within an aortic aneurysm. This example illustrates the tube portion 2 of the graft and attached to some wire 62 or partial catheter within it. Note that the manufacturer can supply the graft with just the tube portion in a very short sheath. This very short sheath 60 could allow the inserter to shorten the graft limbs to the desired length as shown in FIG. 14 and then allow the graft 1 to be inserted directly into an introducer sheath 61.

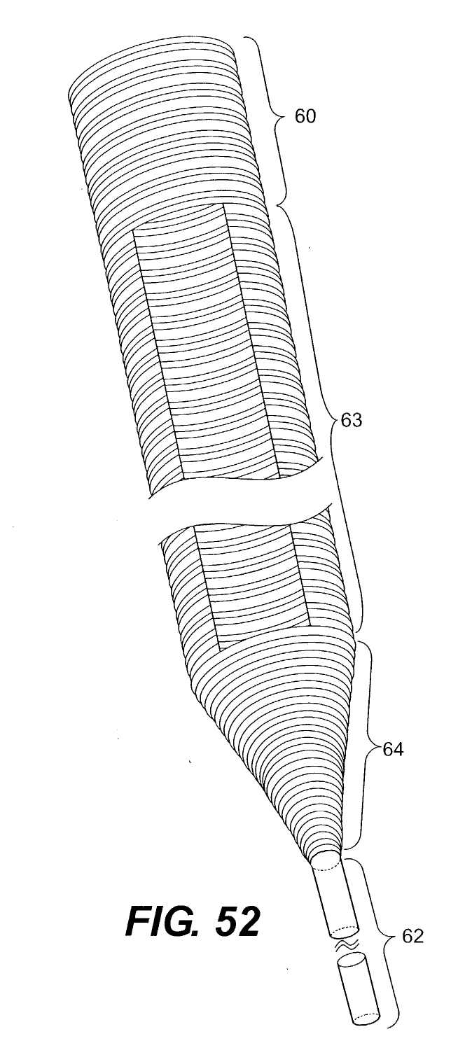

FIG. 52 is another embodiment of a sheath design analogous to that depicted in FIG. 50 though the catheter 62 would most likely have a lumen to allow a mechanism to pass through in order to deploy the graft 1. This is a depiction of closed circumferential sheath 60 that becomes a partial or open sheath 63 that cones down 64 to an open catheter 62.

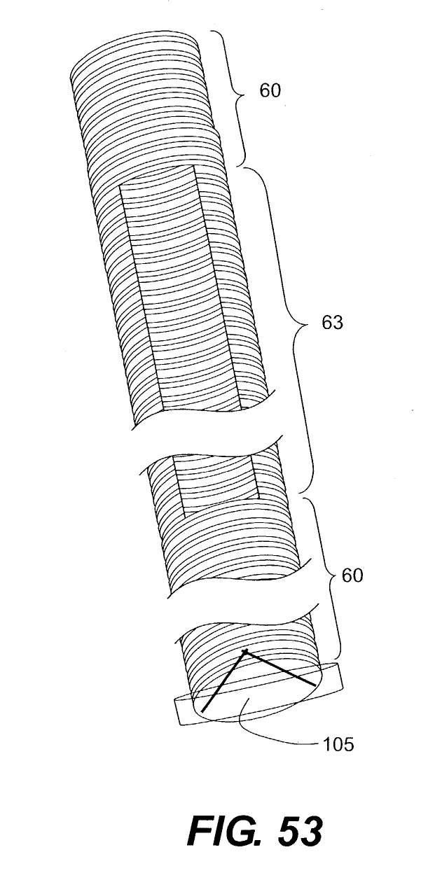

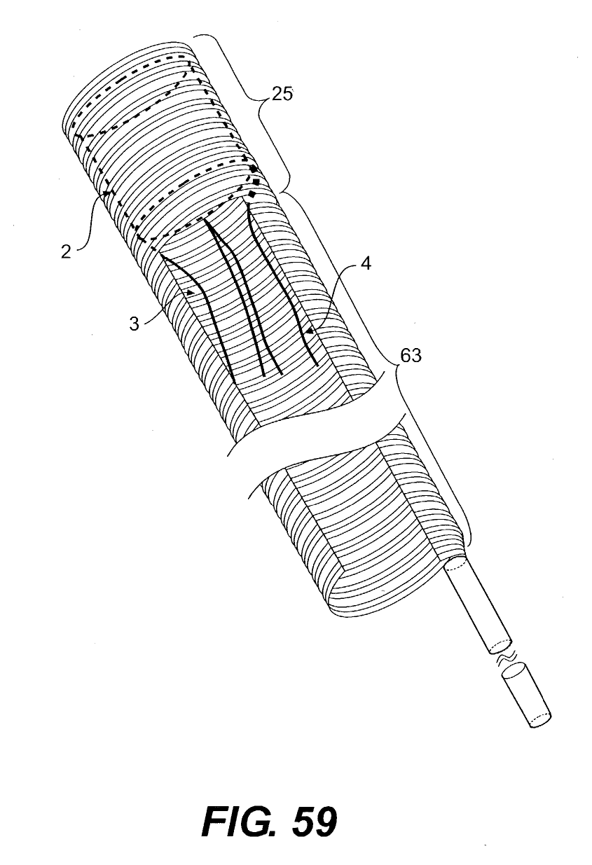

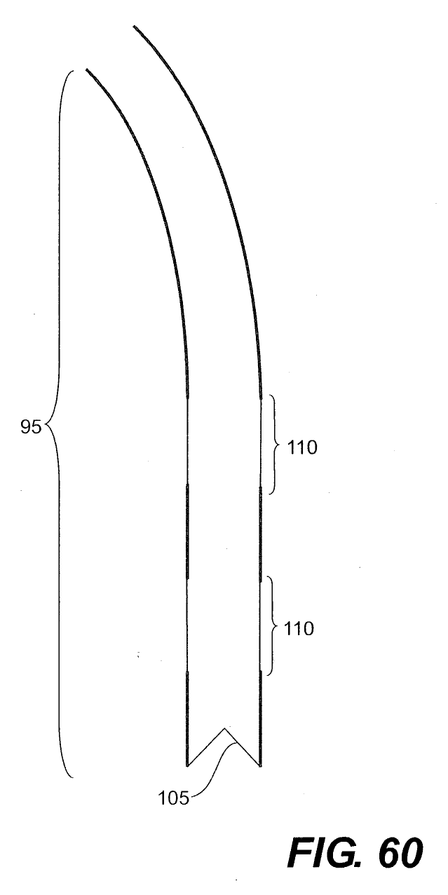

FIG. 53 depicts a closed circumferential sheath 60 that becomes a partial or open sheath 63 that then resumes its closed circumferential sheath configuration 60 that then may end in a hemostatic valve 105. Although not shown, the introducer sheath could be supplied with one or more soft or resilient areas, as depicted in FIG. 59, and the hemostatic valve could be replaced with a connector, as depicted in FIG. 60.

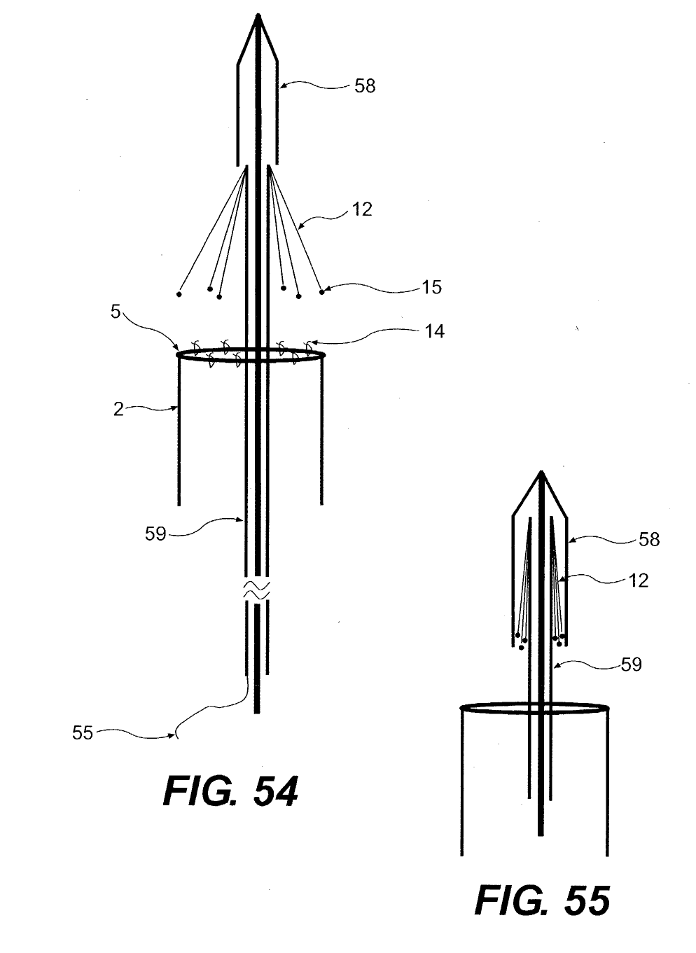

In FIG. 54, an electrical current is applied by an actuator 66 within the catheter 59 when it is desired to release the attachment wires 12 at the small attachment mechanism ball 15 attached to the sutures 14 that are in turn attached to the top 5 of the tube portion 2 of the graft. This release mechanism is also shown in FIGS. 4, 5 and 6. This current triggers the release mechanism. The containment sheath 58 in FIG. 54 is depicted as no longer constraining the attachment wires 12. This method of attachment and release can be used in holding and then detaching any foreign material such as, but not limited to, plastic, fabric, metal, alloy or any combination in any artery or vein or, indeed, in any tissue where a suture or other attachment device is intended to be a temporary (seconds, minutes, hours, days, months, years) attachment mechanism from which subsequent release may be desired. A similar release mechanism could be achieved with any object that changes character when exposed to energy such as light including, but not limited to, infrared and ultraviolet, sound waves, or electricity. Magnets and glues that dissolve upon electrical or heat stimulation or metal alloys that separate in response to heat or electrical current (this includes using resistive heating for activating a heat sensitive release mechanism for any foreign body within any vessel (artery or vein) or within any tissue space can also be used).

FIG. 55 depicts the attachment wires 12 captured within the containment sheath 58 and compressed toward the catheter 59. The constraining and releasing of the attachment wires illustrated is achieved by a push/pull mechanism. This could also be performed by a screwing mechanism, a ratcheting mechanism, or a twisting or turning mechanism.

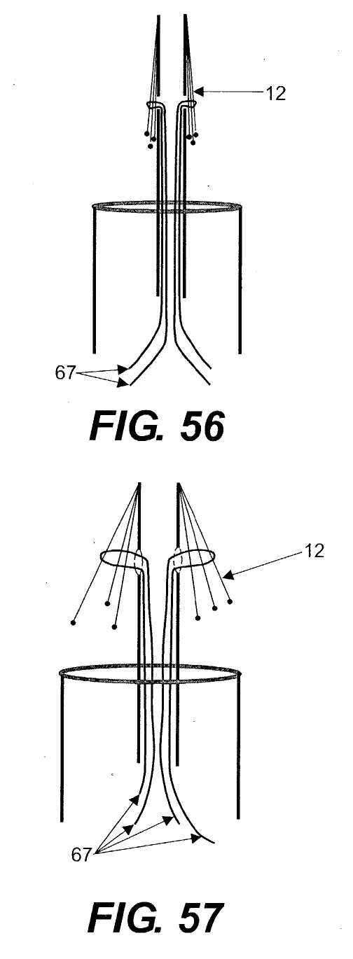

FIGS. 56 and 57 depict another embodiment to constrain (FIG. 56) and release (FIG. 57) the attachment wires 12 with pull wires 67.

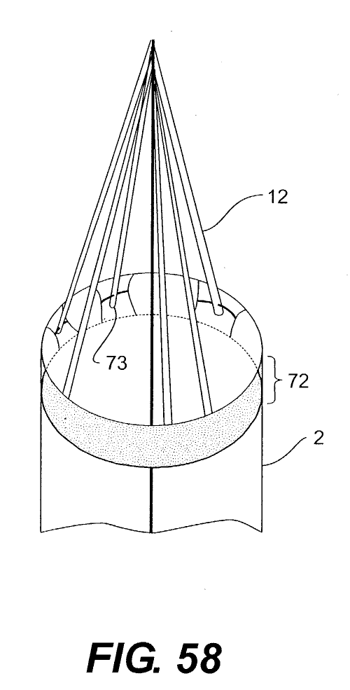

In an alternate embodiment, FIG. 58 depicts a method of attaching and detaching the attachment wires 12 to the tube portion 2 of the graft. The graft is constructed with a semi-rigid circumferential attachment hoop 72 in the neck of the graft 1. The attachment hoop 72 may be composed of, but is not limited to, metal or polymer, or any other suitable material. The hoop serves as an attachment point 73 where the attachment wires 12 connect to splay or contract the graft 1. The attachment wires 12 fasten to the attachment hoop 72 at intervals around the hoop either by the inserter or at the factory before sterilization. The attachment wires 12 may be, but are not limited to, circumferential struts. The struts can be normally splayed as shown, but is not limited to, a diameter of about 10-30 mm or they can be normally axial with the support wire 11 and activated with secondary struts as is familiar in an umbrella opening mechanism. Schemes relating to reversible attaching and detaching are exemplary only and are not limited to those described below.

In one scheme, the attachment hoop is withdrawn from the strut, graft and the patient by an endovascular grasper allowing the struts to assume a position axial with the central catheter.

In a second scheme, the attachment hoop is a polymer that dissolves in blood in a time frame sufficient to carry out the correct placement and fastener attachment.

In a third scheme the attachment point detaches given sufficient force or energy which may include, but is not limited to electrical energy. With electrical energy the attachment point can soften and separate at the attachment points thus releasing the struts and the support wire. With electrical energy the strut end (attachment point) can assume a shape that releases the circumferential hoop.

It should be noted that the attachment hoop is not necessarily linear and can be shaped to be folded and inserted or can be shaped at the attachment points. Additionally, the attachment hoop does not necessarily need to be continuous and can be intermittent and confined to the attachment points.

In a fourth scheme, the attachment hoop is a long flexible strand such as, but is not limited to metal or polymer of a length allowing it to extend to the exterior of the patient. In this scheme the strand is pulled and it releases the struts at the attachment points.

FIG. 59 depicts the tube portion 2 of an graft contained within the short sheath 25 and the graft limbs 3 and 4 not constrained by the partial sheath portion 63 of the combination sheath. By way of example, FIG. 59 illustrates a graft within a sheath similar to that depicted in FIG. 50.

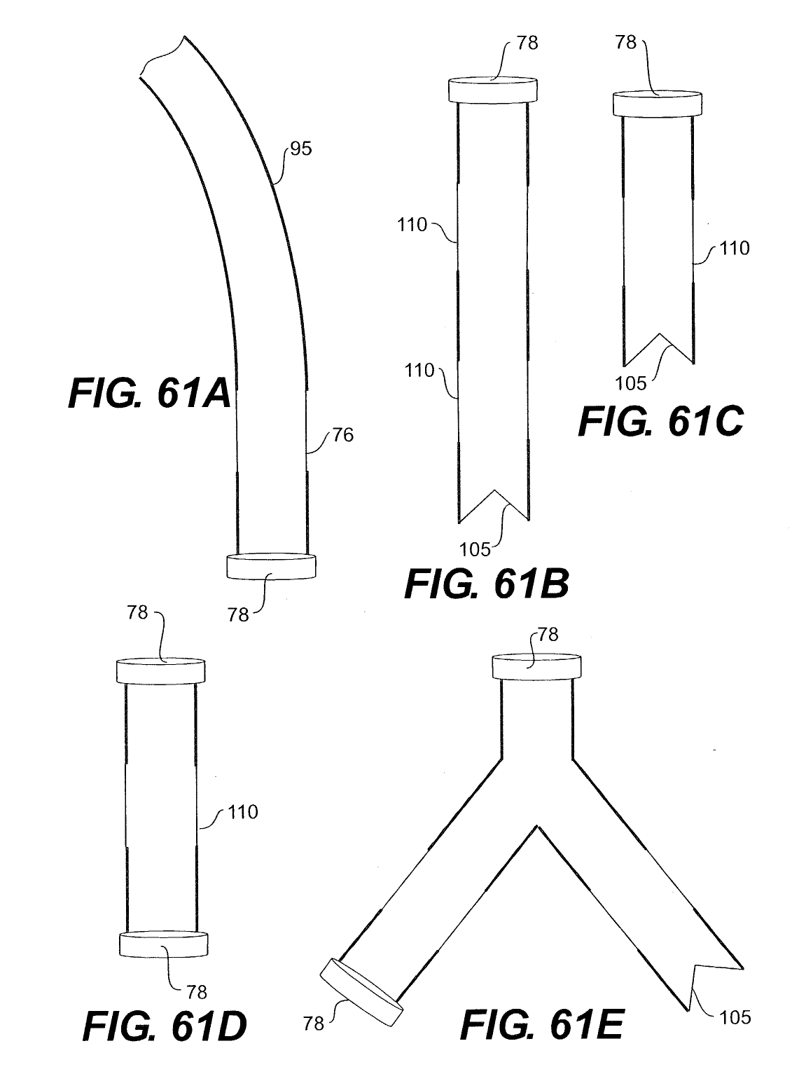

According to FIG. 60, the introducer sheath 95 may be, but is not limited to, about 3 to 30 French (F) (3 F=1 mm) in internal diameter. The introducer sheath 95 can have one or more soft or resilient areas 110 that may remain outside the artery or vein and can be clamped or snared to eliminate or reduce backflow of blood. The soft or resilient areas can be constructed of, but is not limited to, rubber, cloth, plastic or any other suitable material that is relatively compressible. A hemostatic valve 105 is also depicted. This valve can be replaced with a connector 78, as illustrated in FIG. 61.

FIG. 61 depicts a variety of combinations of introducer sheaths 95 allowing the ability to construct, at the time of use, an introducer sheath that meets the needs of the individual inserting objects into a vessel, artery or vein. The basic sheath, FIG. 61A, may or may not have a soft area built in. It does have a connector 78 (snap-on, screw, latch etc.) that provides the ability to connect a variety of extensions or additional ports. FIG. 61B depicts an extender that has two soft areas 110 and a hemostatic valve 105. FIG. 61C has only one soft area 110 and a valve 105 whereas FIG. 61D has a soft area and a connector 78 on each end. FIG. 61E offers the possibility of adding multiple ports. These embodiments are some of the possible variations of connectors, soft areas and hemostatic valves that could be constructed and are intended to be illustrative and exemplary only.

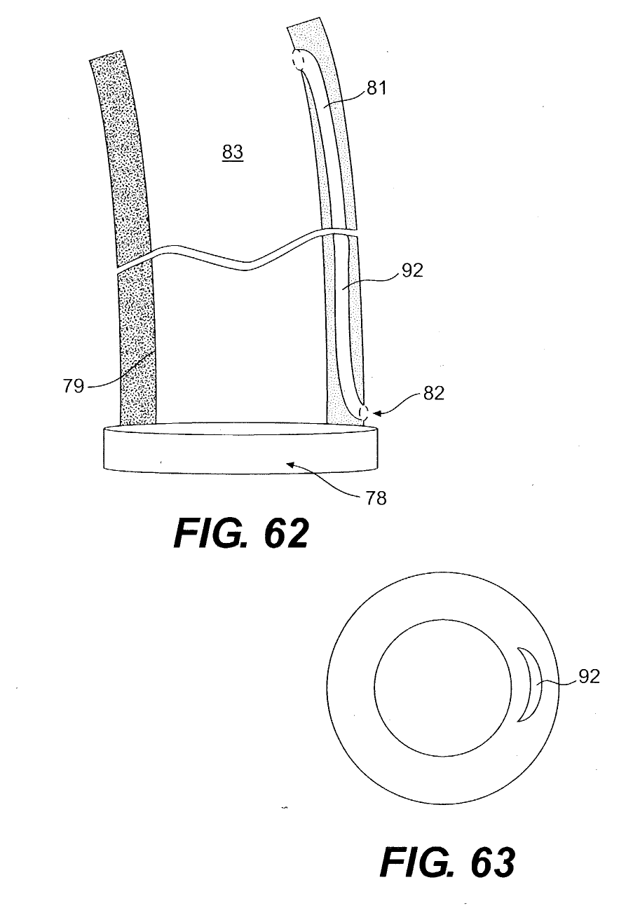

FIG. 62 shows that, within the inner wall 79 of the introducer sheath 95, there can be constructed a small port 81, 82 or multiple ports for the infusion of a liquid such as, but not limited to, a heparin solution. The ports 81, 82 link to a passageway 92 or a plurlity of passageways that may extend the length of the introducer sheath or a predetermined length. The entrance of the port 82 may be near the connector 78 or hemostatic valve 105 outside the area of insertion into the artery or vein. The ports exit site(s) may be near the top of the introducer sheath into the lumen 83 or multiple exit points at discreet places along the length or continuously along the length from proximal to distal. The method of distributing the infusate distally can be via a lumen in the internal diameter, in the wall or on the outside of the sheath wall via a secondary lumen.

The introducer sheath may have a simple sheath with a side infusion port and hemostatic valve or a simple sheath with a side infusion port and snap-on connector or sheath with soft area with infusion port or any combination thereof. FIG. 63 is a cross-section of the introducer sheath showing the infusion port 81.

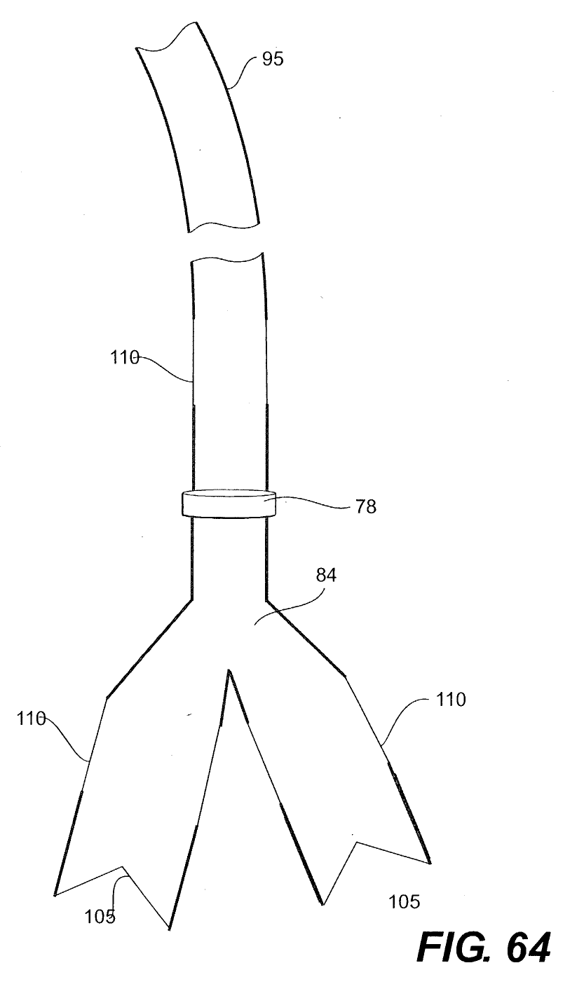

FIG. 64 depicts an introducer sheath 95 containing one soft spot 110 with a connector 78 attached to a double port 84. Each has a soft spot 110 and a hemostatic valve 105. FIG. 64 demonstrates how the connectors work and also shows that the lumens of the add-on ports and extenders can be of varying diameters.

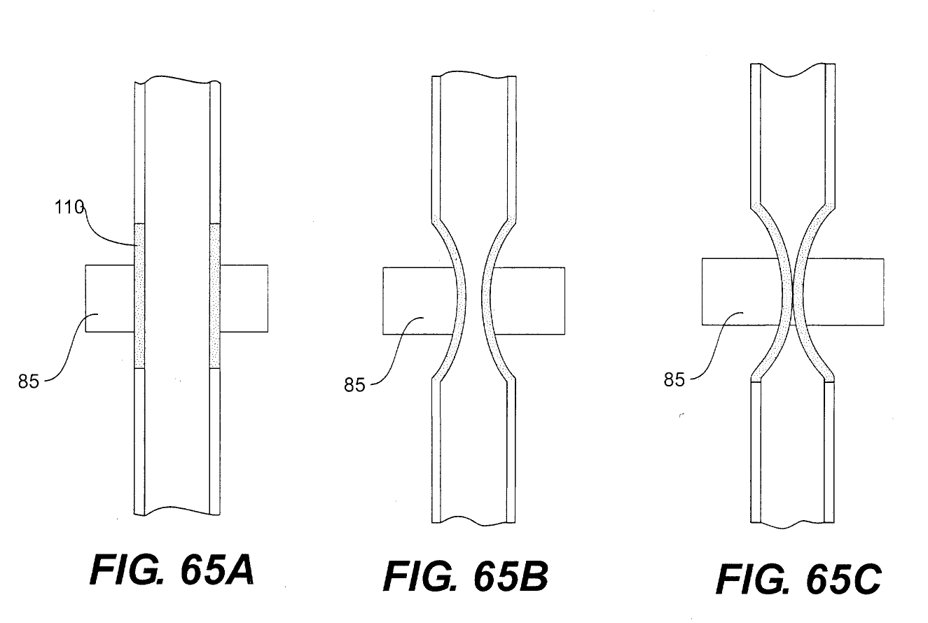

FIG. 65 depicts another possible feature of the introducer sheath 95 with a soft or resilient section 110, also illustrated in FIG. 61. This is a user-controlled variable restricting device 85 that is in circumferential contact with the resilient section 110. The user-controlled variable restricting device 85 is adjustable for the desired internal aperture of the resilient section. The desired internal aperture can be fully open for the largest outer diameter catheter A, partially open for hemostasis around any size catheter (including a guide wire) B, or fully closed for total hemostasis C. The purpose of the device is aperture control and thereby accommodating most sizes of devices passing through the internal diameter of the introducer sheath.

An advantage of alternative embodiments of the invention depicted in FIGS. 66 and 67 is to cut a suture along its length at a location remote from the operator and for the device to track over a suture or along side of it by a suture cutter 80. FIG. 66 shows a catheter based device 80 which tracks over a suture 14 to a remote location and cuts the suture at the distal end of the catheter when the operator inputs mechanical energy into the mechanical actuator 86. In this example the mechanical actuator 86 is pushed and the cutter 87 moves through the suture 14 and cuts it in two pieces 88 and 89. When the mechanical actuator 86 is withdrawn the counter-resistance mechanism 90 such as, but not limited to, spring, flex band as shown, or any other suitable counter-resistance mechanism causes the cutter 87 to return to its original position. Alternatively a spring can act as the force driving the cutter through the suture when the mechanical actuator is withdrawn. Also, the actuator and cutter can be arranged so that rotational or linear movement will cause the cutter to pass through the suture and cut it. An alternative method of affecting the cut of the suture can be laser light. The source of laser light immediately adjacent to the suture would be an optical fiber or a laser diode.

It is further noted that the suture can be fed through the lumen of the suture cutter via a simple snare. If the suture cutter tracks along side of the suture, the suture can be fed through a shorter lumen either axially or from the side. A side loading design would allow the suture to enter from the side but not allow its exit during the tracking procedure prior to cutting.

It will be apparent to those skilled in the art that variations and modifications of the present invention can be made without departing from the scope or spirit of the invention. For example, the method of performing a surgical procedure could be used in settings other than the repair of aneurysms. The method could be used to attach any prosthetic material to any tissue with a metal or plastic attachment device, such as a shape memory metal, plastic staple, or metal staple. For instance, the method could be used to attach a prosthetic mesh to fascia through a laparoscope/endoscope or directly in an open operation for hernia repair. Thus, it is intended that the present invention cover all such modifications and variations of the invention, provided they come within the scope of the appended claims and their equivalents.

What is claimed is:

1. An insertion apparatus for deploying a surgical component to a site in a vessel comprising:
attachment means for attaching the surgical component to the insertion apparatus, wherein the attachment means is releasably connected to the surgical component; and
release means for releasing the attachment means from the surgical component, wherein the release means is in communication with the attachment means.

2. The insertion apparatus of claim 1, further comprising fastening means for fastening the surgical component to the vessel.

3. The insertion apparatus of claim 1, wherein the surgical component is a graft.

4. The insertion apparatus of claim 1, wherein the release means supplies an electric current to the attachment means.

5. The insertion apparatus of claim 1, wherein the release means supplies heat to the attachment means.

6. The insertion apparatus of claim 1, wherein the surgical component comprises at least one distinctive marker.

7. The insertion apparatus of claim 1, wherein the attachment means comprises at least one attachment wire and a support wire.

8. The insertion apparatus of claim 1, wherein the attachment means comprises an attachment hoop, at least one attachment wire, and a support wire.

9. The insertion apparatus of claim 1, wherein the insertion apparatus further comprises a short sheath.

10. The insertion apparatus of claim 9, wherein the short sheath comprises a flare.

11. The insertion apparatus of claim 1, wherein the surgical component corresponds to the site in the vessel with an appropriate diameter and length.

12. An insertion apparatus for deploying a surgical component to a site in a vessel comprising:
at least one attachment wire with a first end and a second end;
a support wire;
wherein the first end of the at least one attachment wire is releaseably connected to the surgical component and the second end is connected to the support wire; and
wherein the attachment wire and support wire positions the surgical component at a site in the vessel.

13. The insertion apparatus of claim 12, further comprising a fastener delivery catheter.

14. The insertion apparatus of claim 12, wherein the surgical component is a graft.

15. The insertion apparatus of claim 12, further comprising an electric current supplied to the attachment wire for releasing the surgical component.

16. The insertion apparatus of claim 12, wherein the surgical component comprises at least one distinctive marker.

17. The insertion apparatus of claim 12, wherein the insertion apparatus further comprises a short sheath.

18. The insertion apparatus of claim 17, wherein the short sheath comprises a flare.

19. The insertion apparatus of claim 12, wherein the surgical component corresponds to the site in the vessel with an appropriate diameter and length.

20. The insertion apparatus of claim 12, further comprising an introducer sheath.

21. The insertion apparatus of claim 20, wherein the introducer sheath comprises a homostatic valve and a dilator.

22. The insertion apparatus of claim 20, wherein the introducer sheath comprises at least one soft portion.

23. The insertion apparatus of claim 22, wherein the soft portion is a user-controlled variable restricting device wherein the soft portion is adjustable.

24. The insertion apparatus of claim 20, wherein the introducer sheath is a closed circumferential sheath.

25. The insertion apparatus of claim 20, wherein the introducer sheath is an open sheath.

26. An insertion apparatus for deploying a surgical component to a site in a vessel comprising:
an attachment hoop releasably connected to the surgical component;
at least one attachment wire with a first end and a second end;
a support wire;
wherein the first end of the at least one attachment wire is connected to the attachment hoop and the second end of the at least one attachment wire is connected to the support wire; and
wherein the insertion apparatus positions the surgical component in the vessel.

27. The insertion apparatus of claim 26, further comprising a fastener delivery catheter.

28. The insertion apparatus of claim 26, wherein the surgical component is a graft.

29. The insertion apparatus of claim 26, further comprising an electric current supplied to the attachment hoop for releasing the surgical component.

30. The insertion apparatus of claim 26, wherein the surgical component comprises at least one distinctive marker.

31. The insertion apparatus of claim 26, wherein the surgical component further comprises a short sheath.

32. The insertion apparatus of claim 31, wherein the short sheath comprises a flare.

33. The insertion apparatus of claim 26, wherein the surgical component corresponds to the site in the vessel with an appropriate diameter and length.

34. The insertion apparatus of claim 26, further comprising an introducer sheath.

35. The insertion apparatus of claim 34, wherein the introducer sheath comprises a detachable homostatic valve and a dilator.

36. The insertion apparatus of claim 34, wherein the introducer sheath comprises at least one soft portion.

37. The insertion apparatus of claim 36, wherein the soft portion is a user-controlled variable restricting device wherein the soft portion is adjustable.

38. The insertion apparatus of claim 34, wherein the introducer sheath is a closed circumferential sheath.

39. The insertion apparatus of claim 34, wherein the introducer sheath is an open sheath.

40. An insertion system for deploying a surgical component to a site in a vessel comprising:
a surgical component;
an insertion apparatus;
attachment means for attaching the surgical component to the insertion apparatus, wherein the attachment means is releasably connected to the surgical component; and
release means for releasing the attachment means from the surgical component, wherein the release means is in communication with the attachment means.

41. The insertion system of claim 40, further comprising a fastening means for fastening the surgical component to the vessel.

42. The insertion system of claim 40, further comprising an introducer sheath.

43. The insertion system of claim 42, wherein the introducer sheath comprises a homostatic valve and a dilator.

44. The insertion system of claim 42, wherein the introducer sheath comprises at least one soft portion.

45. The insertion system of claim 44, wherein the soft portion is a user-controlled variable restricting device wherein the soft portion is adjustable.

46. The insertion system of claim 42, wherein the introducer sheath is a closed circumferential sheath.

47. The insertion system of claim 42, wherein the introducer sheath is an open sheath.

48. The insertion system of claim 40, further comprising a fastener delivery catheter.

49. The insertion system of claim 40, wherein the surgical component is a graft.

50. The insertion system of claim 40, wherein the surgical component comprises at least one distinctive marker.

51. The insertion system of claim 40, wherein the insertion apparatus further comprises a short sheath.

52. The insertion system of claim 51, wherein the short sheath comprises a flare.

53. The insertion system of claim 40, wherein the surgical component corresponds to the site in the vessel with an appropriate diameter and length.

54. The insertion system of claim 40, wherein the surgical component comprises at least one distinctive marker.

55. The insertion system of claim 40, wherein attachment means comprises at least one attachment wire and a support wire.

56. The insertion system of claim 40, wherein attachment means comprises an attachment hoop, at least one attachment wire, and a support wire.

57. The insertion system of claim 40, wherein release means comprises an electric current supplied to attachment means for release of the attachment means from the surgical component.

58. A method for positioning a surgical component to a site in a vessel comprising the steps of:
introducing an insertion apparatus proximal to the site in the vessel;
activating the insertion apparatus; and
withdrawing the insertion apparatus.

59. The method of claim 58, further comprising the step of inserting an introducer catheter to the site in the vessel prior to introducing the insertion apparatus to the site in the vessel.

60. A method for positioning a surgical component with an insertion apparatus to a site in a vessel comprising the steps of:
introducing the insertion apparatus proximal to the site in the vessel;
fastening the surgical component to the vessel;
activating the insertion apparatus; and
withdrawing the insertion apparatus.
